(19) United States
(12) Patent Application Publication
(10) Pub. No.: US 2002/0143358 A1
(43) Pub. Date: Oct. 3, 2002

(54) Method and apparatus for micro-dissection of vascular occlusions

(76) Inventors: Nicanor A. Domingo, Brisbane, CA (US); Robert K. Deckman, San Bruno, CA (US); Brent D. Seybold, Santa Clara, CA (US); Kurt D. Sparks, Palo Alto, CA (US)

Correspondence Address: PERKINS COIE LLP, P.O. BOX 2168, MENLO PARK, CA, 94026, US

(21) Appl. No.: 10/074,546

(22) Filed: Feb. 12, 2002

Related U.S. Application Data
(60) Provisional application No. 60/268,654, filed on Feb. 13, 2001.
(60) Provisional application No. 60/268,647, filed on Feb. 13, 2001.
(60) Provisional application No. 60/268,652, filed on Feb. 13, 2001.
(60) Provisional application No. 60/268,655, filed on Feb. 13, 2001.

(51) Int. Cl. A61B 17/00

(52) U.S. Cl. 606/190; 606/207

(57) ABSTRACT

A method and apparatus for micro-dissection of vascular occlusions are provided wherein two or more tissue expansion members are coupled with a base section and an actuation assembly so that they rotate radially outward from the central axis of the base. The actuating assembly occupies a channel within the base such that it is free to move in a longitudinal direction. As an external force is applied to the actuation assembly, the actuation assembly engages the tissue expansion members causing them to move in a radial outward direction with respect to the base section. The resulting motion causes the tissue expansion members to contact the tissue walls and/or the occlusion. The tissue expansion members can stretch the tissue walls causing the occlusion to tear, fracture or be disrupted or displaced.

### CROSS REFERENCE TO RELATED APPLICATIONS

This application claims the benefit of U.S. Provisional Patent Application Nos. 60/268,654, 60/268,647, 60/268,652, and 60/268,655, all filed Feb. 13, 2001, which are incorporated herein by reference in their entirety. This application is related to U.S. patent application Ser. Nos. 09/981,526 and 09/981,498, both filed Oct. 16, 2001 which are incorporated herein by reference in their entirety.

### TECHNICAL FIELD

The following disclosure relates to medical devices designed for the treatment of vascular occlusions. More particularly, it relates directed to a tissue expansion apparatus for fracturing, disrupting, or displacing a chronic total occlusion.

### BACKGROUND

Medical science has long sought effective treatments for disease states that cause stenosis, which is a narrowing or obstruction of the interior passageway of an artery or vein. This condition, known generally as a vascular occlusion, can be found in patients suffering from such diseases as atherosclerosis (an accumulation of fibrous, fatty or calcified tissue in the arteries or veins). An occlusion may be partial or total, as well as soft and pliable or hard and calcified. Occlusions may also be found at a great variety of sites in the vascular system including the aorta, vena cava, as well as coronary and peripheral arteries and veins.

One method for treating vascular occlusions has been through the use of bypass surgery. Generally, this is a procedure wherein a segment of a patient's vein may be taken from another area of the body and then grafted onto the affected artery at points proximal (upstream) and distal (downstream) to the occluded segment. Thus, the occlusion is bypassed by a new section of vasculature. While the procedure can be effective at restoring blood flow to the tissue surrounding a total occlusion, it is a major surgical procedure with significant morbidity and mortality risks. Furthermore, this procedure requires a long convalescence period and, since the cause of the occlusion has not been alleviated, the occlusion can reoccur in the grafted vasculature. While subsequent bypass surgeries can be undertaken, the risks associated with such subsequent procedures are elevated from the original procedure.

Newer, minimally invasive procedures are now preferred in the treatment of both total and partial vascular occlusions. These procedures often include the use of long, thin, and highly flexible devices known as catheters. During the procedure, the catheter is introduced into a major artery or vein through a small arterial puncture made in the groin, upper arm, or neck, and is advanced and steered into the site of the stenosis or occlusion. Various devices or working elements can be attached to the distal end of the catheter for operating upon the stenosed artery.

Directional coronary atherectomy (DCA) is one example of a minimally invasive procedure used when the lumen, the interior portion of a vein or artery, is narrowed yet remains functionally open. In a DCA procedure, a catheter containing a cutter housed in the distal end of the catheter is advanced over a previously placed guide-wire into the stenosed vasculature segment. The housing is urged against the constriction by the inflation of a balloon so that part of the narrowed lumen intrudes through a window in the side of the housing. Under fluoroscopic observation, the cutter is used to shave away the obstructive material. The shavings are collected in the nosecone of the housing and withdrawn along with the catheter.

Directional coronary atherectomy, however, is often inefficient, time consuming, and at times dangerous. Furthermore, it can only be utilized where a guide-wire has traversed the stenosed section. DCA is an ineffective procedure for treating a “chronic total occlusion” (“CTO”), or an occlusion which totally blocks the vasculature. As mentioned above, the cutter of the DCA needs to be guided to the area of the constriction over a wire, whereupon the operator of the cutter attempts to shave pieces of the occlusion from the walls of the occlusion. This cannot be done in the presence of a CTO. Other procedures aimed at restoring blood flow through stenosed vasculature are also inhibited by a CTO. Angioplasty, or the inflation of a balloon inside a vessel to expand the volume of the lumen, as well as the implantation of stents, require the vessel be at least partially free of an obstruction. Both of these procedures, as do many others, rely on the existence of a guide-wire placed through the occluded area, over which therapeutic devices are advanced to treat the occluded area.

When conditions are such that a CTO exists, guide wires are typically incapable of traversing the occlusion. In some instances a guide-wire or similar device may traverse around the occlusion, penetrate and remain within the vessel wall, yet be unable to re-enter the true lumen at a site distal to the occlusion. Thus, the inability to establish a guide wire position across the occlusion prevents subsequent use of therapeutic treatments such as angioplasty or stenting.

### BRIEF DESCRIPTION OF THE DRAWINGS

The present invention is illustrated by way of example in the following drawings in which like references indicate similar elements. The following drawings disclose various embodiments of the present invention for purposes of illustration only and are not intended to limit the scope of the invention.

FIG. 1 illustrates one embodiment of a tissue expansion apparatus used for treating vascular occlusions in accordance with one embodiment.

FIG. 2 illustrates an exploded view of an embodiment of a tissue expansion apparatus shown in FIG. 1.

FIG. 3 illustrates a cross-sectional view of an embodiment of a tissue expansion apparatus in the un-engaged position in accordance with one embodiment.

FIG. 4 illustrates a cross-sectional view of an embodiment of a tissue expansion apparatus in the engaged position in accordance with one embodiment.

FIGS. 5-7 show differing perspectives of an embodiment of an application of the tissue expansion apparatus used for treating vascular occlusions in accordance with the teachings of one embodiment.

FIG. 8 shows one embodiment of a tissue expansion apparatus allowing for differing degrees of rotation by the individual tissue expansion members, about a central pivot point.

FIG. 9 shows an isometric view of one embodiment of a tissue expansion apparatus used for treating vascular occlusions.

FIG. 10 shows an embodiment of a tissue expansion apparatus first shown in FIG. 9, where the central axis and axis of rotation of the tissue expansion members are offset.

FIG. 11 illustrates an exploded view of a tissue expansion apparatus in accordance with one embodiment.

FIG. 12 illustrates a cross-sectional view of an embodiment of a tissue expansion apparatus in the un-engaged position.

FIG. 13 illustrates a cross-sectional view of an embodiment of a tissue expansion apparatus in the engaged position.

FIGS. 14 and 15 illustrate embodiments of an application of the tissue expansion apparatus of FIG. 9 used for treating vascular occlusions in accordance with the teachings of one embodiment.

FIGS. 16 and 17 show different views of one embodiment of a tissue expansion apparatus used for treating vascular occlusions.

FIGS. 18A-18C shows exploded views of the embodiment of a tissue expansion apparatus shown in FIGS. 16 and 17.

FIGS. 19 and 20 illustrate differing views of one embodiment of a tissue expansion apparatus used for treating vascular occlusions that includes a lumen traversing the apparatus.

FIGS. 21A-21C illustrate an exploded view of the embodiment of a tissue expansion apparatus shown in FIG. 19.

FIG. 22 illustrates a cross-sectional view of an embodiment of a tissue expansion apparatus in the engaged position.

FIGS. 23A-23C illustrate a tissue expansion apparatus used for treating vascular occlusions in accordance with one embodiment as it traverses a vascular occlusion.

FIG. 24 shows an apparatus for micro-dissection of vascular occlusions in accordance with an embodiment that includes tissue expansion members that are offset from the apparatus' central axis.

FIG. 25 shows the embodiment of FIG. 24 in an open or engage orientation.

FIG. 26 is an embodiment of an apparatus for micro-dissection of vascular occlusion that includes tissue expansion members that are offset from the central axis wherein one tissue expansion member possesses a lumen.

FIG. 27 shows the embodiment of FIG. 26 in an open or engage orientation.

FIG. 28 is an embodiment of an apparatus for micro-dissection of vascular occlusions that includes an offset tissue expansion member possessing a lumen that includes the presence of a guide wire.

FIG. 29 is an embodiment of an apparatus for micro-dissection of vascular occlusions that includes offset tissue expansion members with a lumen in one tissue expansion number that is aligned with the apparatus' central axis.

FIGS. 30 and 31 show the apparatus for micro-dissection of vascular occlusions of FIG. 29 from differing perspectives.

FIGS. 32 and 33 show an embodiment of an apparatus for micro-dissection that includes offset tissue expansion members with opposing recessed areas forming a lumen.

### DETAILED DESCRIPTION

An apparatus and method for treating or disrupting a vascular occlusion, such as a chronic total occlusion, contained within the interior of vasculature (e.g., arterial or venous blood vessels of the heart or peripheral vasculature) is presented in detail below. In a first embodiment, an apparatus for treating or disrupting a vascular occlusion comprises a tissue expansion apparatus that is capable of performing blunt micro-dissection of vascular occlusions so as to tear, fracture, or otherwise disrupt the vascular occlusion. The apparatus comprises two tissue expansion members coupled with a base section and an actuation assembly. The proximal end of the tissue expansion members rotate about a central transverse axis of the base, while the distal portion of the tissue expansion members move radially outward from the central axis of the catheter. Slots in the actuation assembly couple to pins in the tissue expansion members to translate the linear input force of the actuation wire or cable into the radial movement of the tissue expansion members. The actuating assembly generally occupies a channel within the base so that it is free to move in a longitudinal direction.

As an external force is applied to translate the actuation assembly in a proximal direction, the slots in the actuation assembly engage the pins in the tissue expansion members, causing them to move in a radial outward direction with respect to the base section. The resulting motion causes the tissue expansion members to impart on the vascular walls an outward radial force from within the vascular lesion causing the occlusion to tear, fracture, dissect or otherwise be disrupted or displaced. Continued advancement of the catheter using this action can establish a dissected pathway through the occlusion, until the terminal end of the occlusion is reached.

In the following description, numerous specific details (apparatus design, alternative orientation of members, specific methods of applying the device, etc.) provide a thorough understanding of, and enabling description for, embodiments of the invention. In general, this description presents four different embodiments of a tissue expansion device. Combinations and variations of the different embodiments will be appreciated by one skilled in the relevant art. One skilled in the relevant art will also recognize that the invention can be made and practiced without one or more of the specific details, or with other elements, methods, etc. In other instances, well-known structures, materials, or operations are not shown, or are not described in detail, to avoid obscuring aspects of the invention.

In general, brief definitions of several terms used herein are preceded by the term being enclosed within double quotation marks. Such definitions, although brief, will help those skilled in the relevant art to more fully appreciate aspects of the invention based on the detailed description provided herein. Such definitions are further defined by the description of the invention as a whole (including the claims) and not simply by such definitions. The term “occlusion” as generally used herein describes a total constriction or blocking of an arterial or venous vessel. Likewise, “pathway” is a term used throughout the description to describe an unrestricted path through the stenosed vasculature such that a guide-wire or similar device can traverse past the stenosed region without exiting the interior section of the vessel. Throughout this description, the term “device” and “apparatus” should be considered synonymous when referred to an “apparatus” for micro-dissection of vascular occlusions. The term “dissecting” is used to describe the tearing, fracturing, cutting, disrupting or separating condition of an occlusion as brought about by the claimed process through the use of the claimed apparatus.

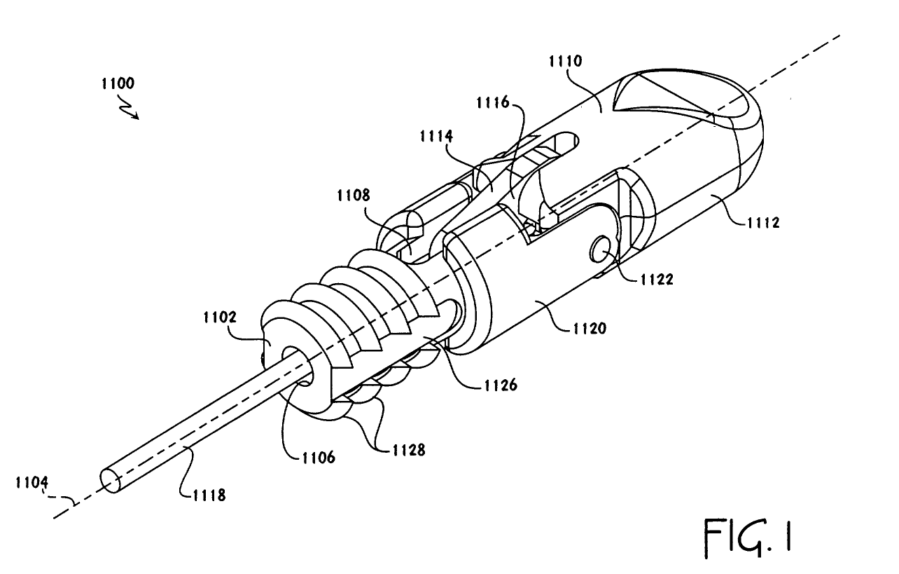

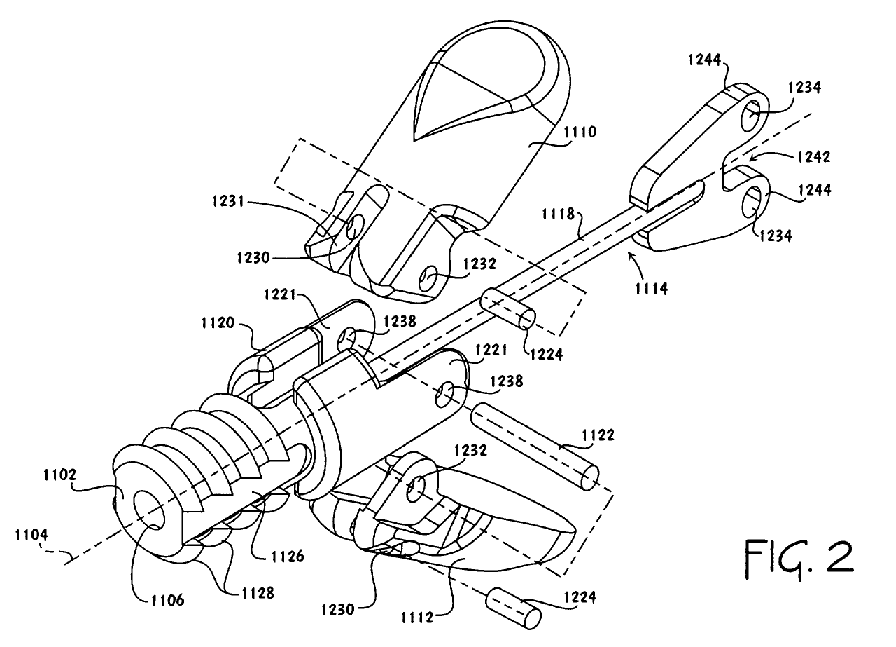

FIG. 1 illustrates a first embodiment of a tissue expansion apparatus 1100 for treating or disrupting a vascular occlusion. FIG. 2 shows an exploded view of the same embodiment. In general, the tissue expansion apparatus described herein can be used to disrupt or dissect a vascular occlusion formed within various interior sections of blood vessels or organs contained in the body. The tissue expansion apparatus is generally located at the distal end of a catheter to enable the positioning of the tissue expansion apparatus to either contact or be in approximate contact with the vascular occlusion and/or a blood vessel wall, in order to initiate a dissected pathway. This pathway can occur within the occlusion itself, between the occlusion and the wall of the vessel, or within the vessel wall itself.

Referring to FIG. 1, the tissue expansion apparatus 1100 includes a base section 1102 having a central axis 1104, base aperture 1106, and an actuation channel 1108. In this embodiment, the tissue expansion apparatus 1100 includes a first tissue expansion member 1110, a second tissue expansion member 1112, an actuation assembly 1114 that includes an actuation plate 1116 and an actuation member 1118, a hinge assembly 1120, a hinge pin 1122 associated with the hinge assembly 1120, coupling pin(s) 1124 which couple each tissue expansion member to the actuation plate 1116, a mounting set channel 1126 and retention fins 1128 to facilitate attachment to the catheter shaft. Other embodiments may include additional or fewer tissue expansion members as well as additional or fewer components. The central axis 1104 extends through the base section 1102 of the tissue expansion apparatus 1100, as well as through the actuating assembly 1114.

In operation, the tissue expansion apparatus 1100 is placed in contact with, or in approximate contact with, a vascular occlusion and/or a blood vessel wall to facilitate the disruption of the vascular occlusion. In this embodiment, an actuation force, including one that is linearly and proximally exerted, is applied to the actuation assembly 1114 (i.e., through actuation member 1118), whereupon the actuation force exerted in a proximal direction is converted into an outward radial force with respect to the hinge assembly 1120. This outward radial force is exerted by the tissue expansion members on or within the occlusion, between the occlusion and the vascular walls, on the vascular walls themselves, or within the vascular walls. In other embodiments, the motion of the tissue expansion members can be chosen to best meet the operating environment. The radial movement of the tissue expansion members can include simultaneous, independent, symmetric, asymmetric, and ratio-metric motion. The spreading or mechanical force exerted by the tissue expansion members (1110, 1112) is applied to the vascular occlusion and/or a blood vessel wall so as to tear, fracture, or otherwise disrupt, the vascular occlusion contained within various sections of vasculature without damaging the blood vessel wall. This methodology is similar to those described in U.S. patent application Ser. No. 09/981,526, filed Oct. 16, 2001, and U.S. patent application Ser. No. 09/984,498, filed Oct. 16, 2001, both of which are currently pending. The continued linear disruption of the vascular occlusion can create a channel or a passageway of sufficient size for the passage of the apparatus and attached catheter shaft until the occlusion has been crossed. Upon retraction of the catheter, a guide-wire or therapeutic catheter can then be advanced within the dissected occlusion for various elective therapeutic procedures.

FIG. 1 further illustrates the tissue expansion apparatus 1100 including a base section 1102 and the tissue expansion members (1110, 1112) coupled to the hinge assembly 1120. The tissue expansion members (1110, 1112) move radially outward with respect to the central longitudinal axis of the hinge assembly 1120. The base section 1102 of the tissue expansion apparatus 1100 includes a base aperture 1106 and an actuation channel 1108, which can accommodate the actuation member 1118 and the actuation plate 1116.

As shown in FIG. 2, the tissue expansion members (1110, 1112) include coupling pin apertures 1230 and hinge member apertures 1232. The hinge member apertures 1232 can accommodate a hinge pin 1122, which couples the tissue expansion members to the hinge assembly 1120, such that the respective tissue expansion members can rotate about the hinge pin 1120 and the distal end moves radially with respect to the hinge assembly 1120. The coupling pin apertures 1230 accommodate coupling pins 1224, which couple the tissue expansion members to the actuation plate 1116 via the corresponding actuation plate apertures 1234. The coupling between the actuation plate 1116 and the respective tissue expansion members (1110, 1112) allows for the conversion of a linear actuation force applied to the actuation member 1118 in a proximal direction, into the radial motion of the respective tissue expansion members (1110, 1112) around the hinge pin 1122.

FIG. 2 shows the base section 1102 of the tissue expansion apparatus 1100 first introduced in FIG. 1. A hinge assembly 1120 is, in this embodiment, affixed to or integrally molded with, the distal region of the base section 1102. The distal region includes the distal face and surrounding area of the base section 1102. In this embodiment, the hinge assembly 1120 comprises two hinge assembly arms 1221 extending longitudinally and parallel to the central axis 1104. The arms 1221 are coupled to the distal face of the base section 1102 and further extend the actuation channel 1108. The hinge assembly arms 1221 also include two hinge assembly apertures 1238 that accommodate a hinge pin 1122. The hinge pin 1122 can engage the hinge member apertures 1232 of the respective tissue expansion member (1110, 1112) and the hinge assembly aperture 1238, coupling the respective tissue expansion member to the hinge assembly arms 1221. The single hinge pin 1122 is perpendicular to and passes through the central axis 1104 providing an axis and center of rotation by which the tissue expansion members (1110, 1112) can move radially outward and inward. The respective tissue expansion member (1110, 1112) can be coupled to the hinge assembly 1120 via the hinge pin 1122 using standard bonding or coupling techniques (laser welding, adhesive, resistance welding, as examples) readily known to one skilled in the relevant art, allowing for their radial movement. In this process, the hinge pin 1122 is affixed to each hinge assembly aperture 1238, and each tissue expansion member 1110, 1112 is free to rotate about the hinge pin 1122 via aperture 1232.

A variety of different configurations can be employed to provide for the functionality of the hinge assembly 1120, such that a axis is provided about which the tissue expansion members (1110, 1112) can rotate. Furthermore, the respective tissue expansion members (1110, 1112) can be made from a variety of, and/or combination of, different materials including, but not limited to, stainless steel, nickel titanium, other shape memory alloys, ceramics, bio-compatible medical plastics, and other materials that are known to those skilled in the relevant art.

The respective coupling of the hinge pin 1122 and coupling pin 1224 to the different components of the tissue expansion apparatus 1100 may be implemented in a variety of ways, such as physical bonding, adhesive bonding, metal joining methods, or other methods which are also well known in the art. For instance, the different components of the tissue expansion apparatus 1100 can be bonded with an epoxy or other appropriate material should the tissue expansion members be made of a polymer. Welding, soldering, brazing, or other methods appropriate for bonding metallic components can also be used. In one embodiment, the coupling of the hinge pin 1122 and coupling pin 1224 to the different components of the tissue expansion apparatus 1100 can be implemented through spot welds, so as to retain the hinge pin's 1122 and coupling pin's 1224 functionality (e.g., rotational freedom) with the different components of the tissue expansion apparatus 1100.

In this embodiment, the actuation assembly 1114 comprises an actuation member 1118, such as an actuation cable, rod or wire for example, coupled to an actuation plate 1116. The actuation member 1118 can be coupled to the actuation plate 1116 by a variety of commonly employed techniques, such as physical bonding, adhesive bonding, and metal joining methods, or other techniques which are well known in the art and commonly employed for bonding or coupling two elements together. In one embodiment, the actuation member 1118 may be configured to limit the amount of longitudinal force applied to the vasculature through the tissue expansion members (1110, 1112) as well as limit the distance of travel of the tissue expansion members. For instance, a pre-selected amount of force can be established such that a force applied in excess of the selected limit will merely deform the actuation member 1118 and will not be transmitted to the blood vessel through the tissue expansion members (1110, 1112). One method of implementation is through the use of a nickel-titanium member. The member can be processed such that up to a specified input tensile force, the longitudinal strain is very small, and has a roughly linear relationship to the input force. Upon exceeding the specified tensile force, the member demonstrates a recoverable strain (elongation) over which the force in the member remains constant.

The actuation plate of this embodiment 1116 contains two actuation plate apertures 1234, a channel section 1242, and outer lobe sections 1244. The two respective apertures 1234 of the actuation plate 1116 are configured to engage the respective coupling pins 1224 which are inserted into the coupling pin apertures 1230 of the respective tissue expansion members (1110, 1112). The tissue expansion members 1110 and 1112 are coupled to the actuation assembly 1114 via the respective coupling pins 1224 and actuation plate 1116, such that an actuation force applied in a proximal direction to the actuation assembly 1116 (e.g., through the actuation member 1118) is converted into a spreading or mechanical force (e.g., radial force with respect to the hinge assembly 1120) exerted on the vasculature by the expansion members. In one embodiment, the tissue expansion members (1110, 1112) can be configured to couple with the actuation assembly 1114 so that the resultant motion of the tissue expansion members (1110, 1112) upon actuation assembly movement is both simultaneous and equal in range and angular movement.

The base section 1102 and the hinge assembly 1120 provide an actuation channel 1108 that supports movement of the actuation plate 1116. In the embodiment shown, the actuation plate 1116 resides and moves within the space defined by the actuation channel 1108. In other embodiments the configuration may differ such that the functuality of the respective components remain the same. As shown in FIG. 2, the actuation member 1118, which is coupled to the actuation plate 1116, passes through the base aperture 1106 of the base section 1102. The interior of the base aperture 1106, as well as the exterior of the actuation member 1118 can be coated with Teflon® or a similar substance, such that any friction from the movement of the actuation member 1118 through the base aperture 1106 is greatly reduced. Thus, an actuation force applied to the actuation member 1118 is efficiently conveyed through the actuation plate 1116 and coupling pins 1224 to the tissue expansion members (1110, 1112), causing the tissue expansion members (1110, 1112) to rotate about the hinge pin 1122.

In an embodiment illustrated in FIG. 2, each tissue expansion member includes a radial channel or member channel 1231. This channel allows each tissue expansion member to accommodate the lobe sections 1244 of the actuation plate 1116 as the respective tissue expansion member rotates about the hinge assembly 1120.

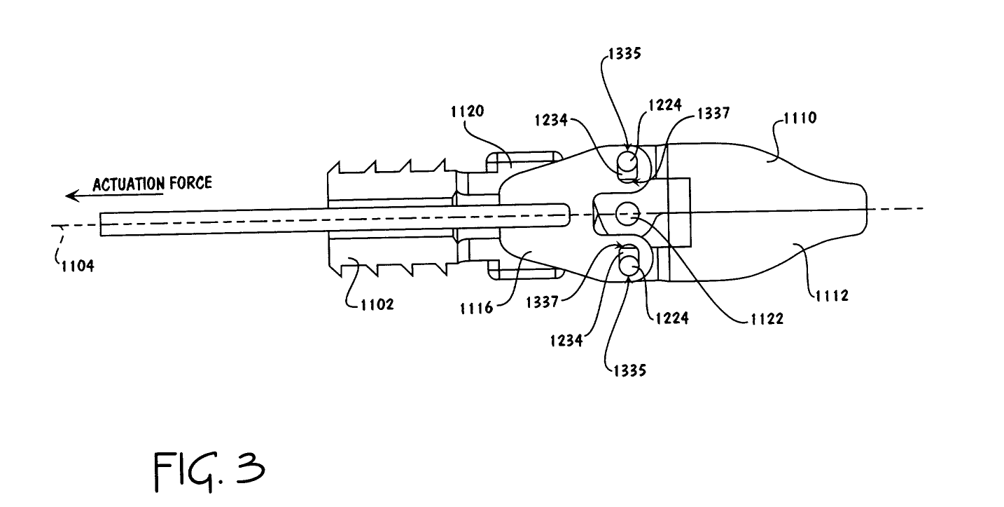

FIG. 3 shows a side cross-sectional view of an embodiment of the tissue expansion apparatus 1100 with the expansion members in a closed or un-engaged position. The tissue expansion members (1110, 1112) are coupled to the hinge assembly 1120 via the hinge pin 1122. As previously described, the longitudinal axis of the hinge pin 1122 defines a central axis about which the expansion members can move radially outwards relative to the hinge assembly 1120. Additionally, the two apertures 1234 of the actuation plate 1116 are configured to engage the respective coupling pins 1224 that are inserted into the coupling pin apertures 1230 of the tissue expansion members (1110, 1112). The actuation plate 1116, in combination with the coupling pins 1224, transfers the external actuation force applied to the actuation member 1118 to the expansion members, allowing for the radial motion of the tissue expansion members (1110, 1112) around the hinge pin 1122. As the tissue expanding members (1110, 1112) rotate about the hinge pin 1122 to an open position, the coupling pins 1224 move both proximally along the central axis 1104 and radially inward toward the central axis 1104. Accordingly, the two actuation plate apertures 1234 of the actuation plate are elongated perpendicular to the central axis 1104 to allow the coupling pins 1224 to move in this direction normal to the central axis 1104 as the expansion members rotate. Thus, the actuation plate apertures 1234 are typically oval in shape but are not so limited. In an alternative embodiment (not shown) the coupling pins can be fixed to the actuation plate and translate within an oval or similarly shaped slot in the expansion members.

When the tissue expansion members (1110, 1112) are in the closed or un-engaged position, the coupling pins 1224 associated with the tissue expansion members are positioned toward the outer sections or edges 1335 of the two apertures 1234 of the actuation plate 1116. As an actuation force is applied to the actuation member 1118 in a proximal direction, the apertures 1234 associated with the actuation plate 1116 engage the respective coupling pin 1224 resulting in the radial motion of the respective tissue expansion member around the hinge pin 1122. As the tissue expansion members (1110, 1112) rotate around the hinge pin 1122, the coupling pins 1224 translate their position toward the inner sections or edges 1337 of the two apertures 1234 of the actuation plate 1116.

In another embodiment of the tissue expansion apparatus 1100, the tissue expansion members (1110, 1112) remain in a closed or un-engaged position until a force is applied to the actuation member 1118 in a proximal direction causing the expansion members to rotate outward. The tissue expansion members (1110, 1112) then return to a closed or un-engaged position either actively by providing a distally directed force manually or through a spring mechanism into the actuation member 1118, or passively by the removal of the actuation force being applied to the actuation member 1118. The tissue expansion apparatus 1100 can be configured biased to the open or engaged position via a spring or similar mechanism. In this alternative embodiment, a distal force of the actuation member 1118 closes the tissue expansion member.

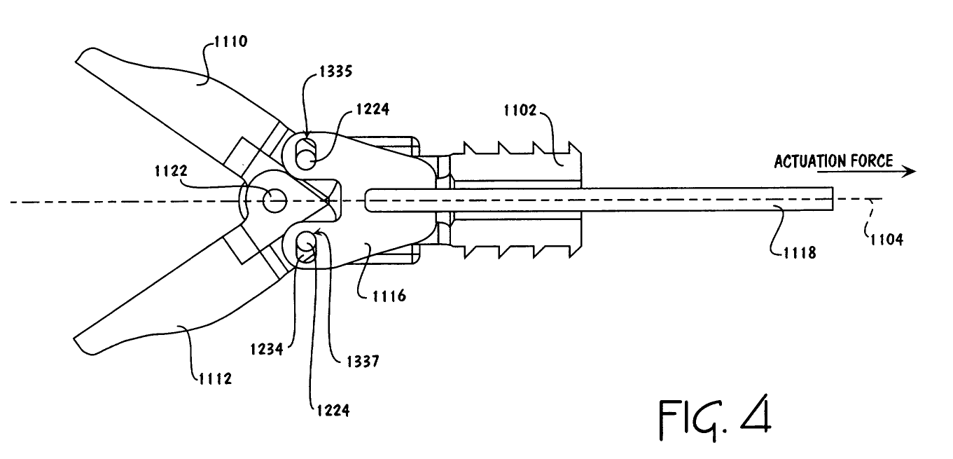

FIG. 4 shows a side cross-sectional view of an embodiment of the tissue expansion apparatus 1100 including two tissue expansion members with the first tissue expansion member 1110 and second tissue expansion member 1112 in the open or engaged positions. When the tissue expansion members (1110, 1112) are in the open or engaged position in response to an actuation force being applied to the actuation member 1118, the respective coupling pin 1224 associated with the tissue expansion members are positioned toward the inner sections 1337 of the two apertures 1234 of the actuation plate 1116. From a closed position, as a force is applied, the coupling pins 1224 shift from the outer sections 1335 of the two actuation plate apertures 1234 toward the inner sections 1337 of the two actuation plate apertures 1234. Correspondingly, in response to the actuation force being applied to the actuation member 1118, the actuation plate 1116 moves within the actuation channel 1108. The resulting motion of the actuation plate 1116, in combination with the coupling pins 1224, transfers the external force to the tissue expansion members (1110, 1112), causing their radial motion around the hinge pin 1122.

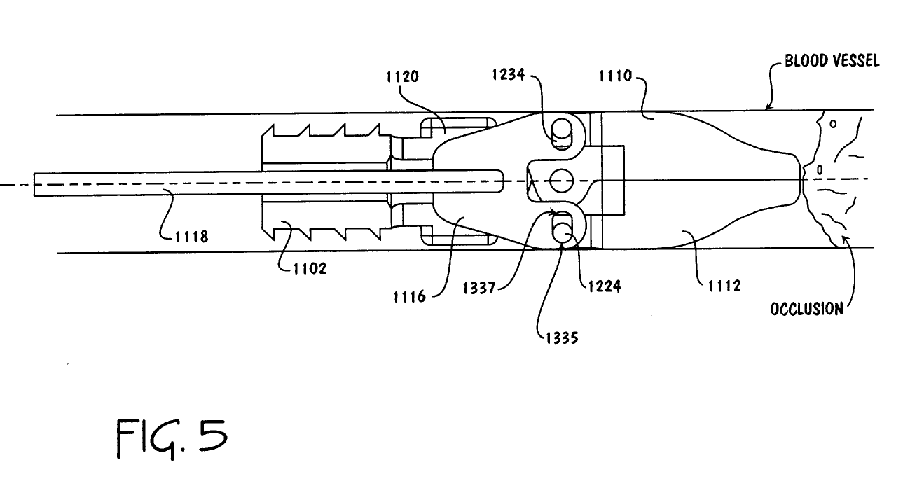

FIG. 5 shows an embodiment of the tissue expansion apparatus 1100 that is placed adjacent to or in contact with a vascular occlusion formed within a section of vasculature. Once the tissue expansion apparatus 1100 is placed into approximate intimate contact with an occlusion, an actuation force is applied to the actuation member 1118, whereupon the respective tissue expansion members (1110, 1112) move radially outwards with respect to the hinge pin 1122. This radial motion or movement of the tissue expansion members tears, fractures, or otherwise disrupts the body of the occlusion internally, between the vessel wall and the occlusion, or within the vessel wall itself. Repetitive application of this process is performed as required to tear, fracture or dissect the occlusion to define a pathway through the occlusion. Once a pathway is established, a guide-wire can be introduced followed by intervention devices or other catheters.

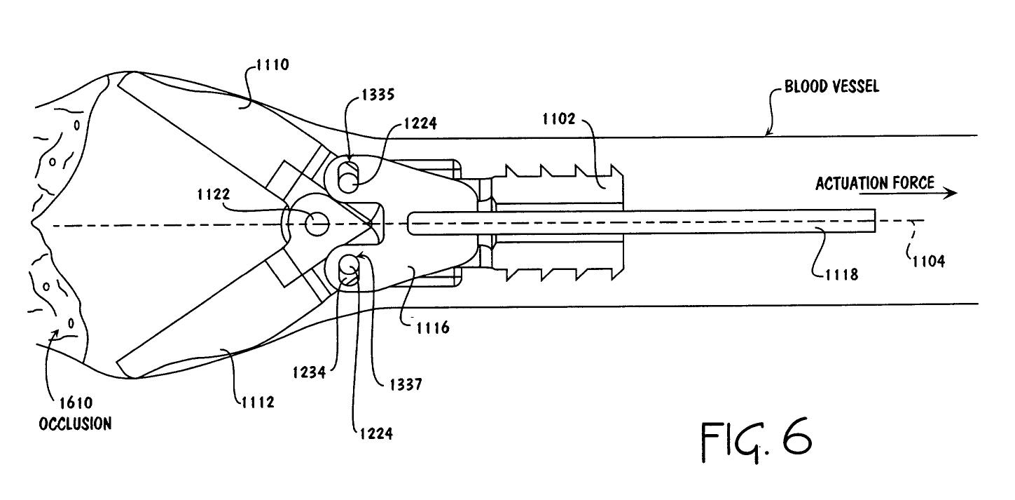

FIG. 6 shows one embodiment of an application of the first embodiment of the tissue expansion apparatus 1100, where the outward radial movement of the tissue expansion members (1110, 1112) tears, fractures, or otherwise disrupts the body of the occlusion 1610, in response to an actuation force being applied to the actuation member 1118. In a further embodiment, the tissue expansion apparatus 1100 can be placed into approximate intimate contact with the occlusion, so that the radial movement of the respective tissue expansion members tears or fractures the body of the occlusion away from the interior lining of an arterial or venous blood vessel in response to the actuation force being applied to the actuation member 1118. As a result of the motion of the tissue expansion members, the blood vessel wall can be stretched to create a path substantially between the occlusion and the blood vessel wall, or within the vessel wall itself. Similarly, when a vascular occlusion is adhered to the wall of the blood vessel, the tissue expansion members can spread apart or fracture the separate layers of the occlusion itself.

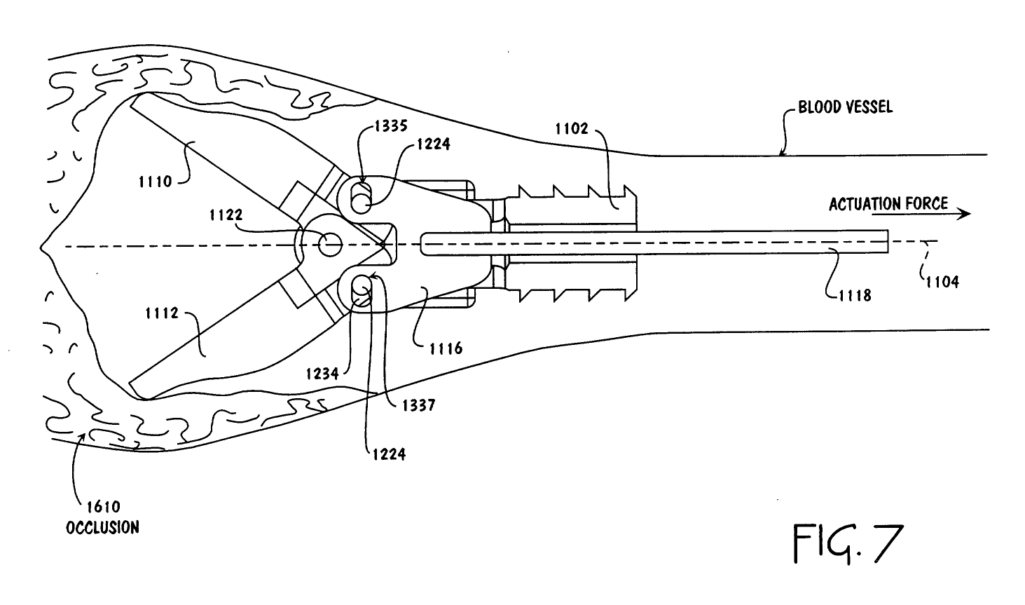

FIG. 7 shows a further application of an embodiment of the expansion apparatus 1100. As the tissue expansion members (1110, 1121) of the tissue expansion apparatus 1110 disrupt the occlusion 1610, the members can retract and return to their closed, unengaged position, forming a smooth uninterrupted profile, allowing the catheter to advance into the dissection track produced by the previous opening of the tissue expansion members 1110, 1112. Once advanced distally into this dissection track, the tissue expansion members (1110, 1112) are again rotated radially outward via a proximal input force to the actuation member 1118, creating another incremental dissection track distal to the last. The tissue expansion members are again returned to a closed, unengaged position, allowing the catheter to be advanced into the new distal portion of the dissection track. This process is repeated until the catheter is advanced through the occlusion and the terminal side of the occlusion is reached. Once through the occlusion, a guide-wire or similar device can be placed in the stenosed vessel to guide other devices.

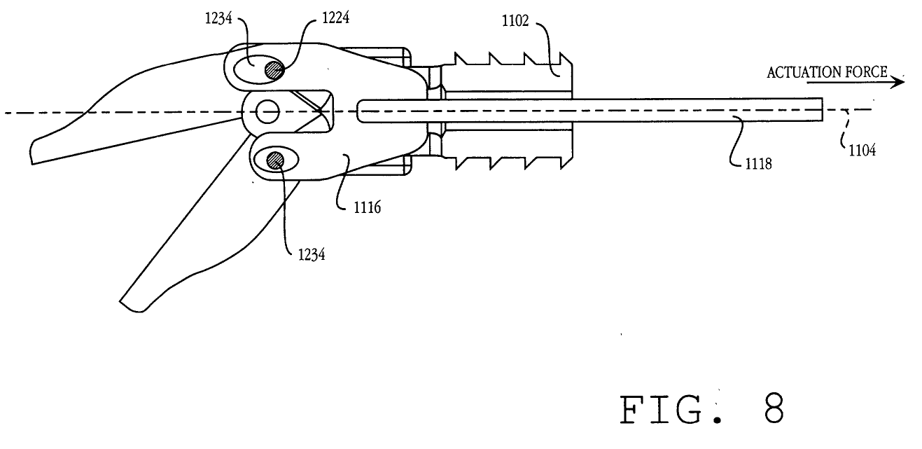

Another aspect of the tissue expansion apparatus, shown in FIG. 8, allows the individual tissue expansion member (1110, 1112) to travel through differing degrees of rotation and to rotate asymmetrically. To accomplish this, the length and/or shape of one of the apertures 1234 of the actuation plate 1116 can be varied with respect to their other aperture. By varying the relative locations of the distal and proximal edges of the apertures 1234, the coupling pins 1224 will engage the edges of the apertures 1234 independently, dependent only upon the translational position of the actuation plate 1116. Thus, each tissue expansion member will begin to rotate at different displacement positions of the actuation plate 1116 and will continue to rotate through different degrees of rotation. As an example to illustrate this principle, (now shown) if both apertures 1234 were of identical rectangular or ovoid shape but one aperture 1234 was orientated axially inline with the proximal direction of movement of the actuation plate 1116 and the opposing aperture 1234 was oriented perpendicular to the proximal motion of the actuation plate 1216, only the tissue expansion member associated with the perpendicular aperture 1234 would rotate as the actuation plate 1116 is moved in a proximal direction. The coupling pin 1224 of the tissue expansion member associated with the axially aligned aperture 1234 would never engage the actuation plate and the tissue expansion member would remain undisturbed. This example is only for descriptive purpose. In actual practice the relative engagement positions of the distal and proximal edges of each aperature would be adjusted to allow each tissue expanding member to open and close through some pre-determined rotational translation. By altering the distal and proximal boundaries as well as the position of the aperture/coupling pin combination, the degree of rotation and the priority of which tissue expansion device moves first can be altered.

The relative positions of the proximal edges of the apertures 1234 can also determine the orientation of the closed position of the tissue expansion members (1110, 1112). The tissue expansion member associated with the aperture having a more distally positioned proximal edge relative to the other tissue expansion member can achieve an un-engaged or closed position beyond the central axis 1104, while the tissue expansion member associated with the aperture having a more proximally positioned proximal edge relative to the other tissue expansion member will achieve its un-engaged or closed position short of the central axis 1104. Likewise, the relative positions of the distal edges of the apertures 1234 will determine the orientation of the open position of the tissue expansion members (1110, 1112). The tissue expansion member associated with the aperture having a more distally positioned distal edge will assume a lesser rotated open or engaged position with respect to the central axis 1104, while the tissue expansion member associated with the aperture having a proximally positioned distal edge will assume a greater rotated engaged position with respect to the central axis 1104. Alternatively, the distal-proximal positions of the coupling pins may be varied such that they independently engage the proximal and distal edges of the apertures at different translational positions of the actuation plate 1116. Collectively, a combination of engagement pin position and aperture 1234 shape may be employed to achieve the desired unique opened and closed positions of each tissue expansion member (1110, 1112).

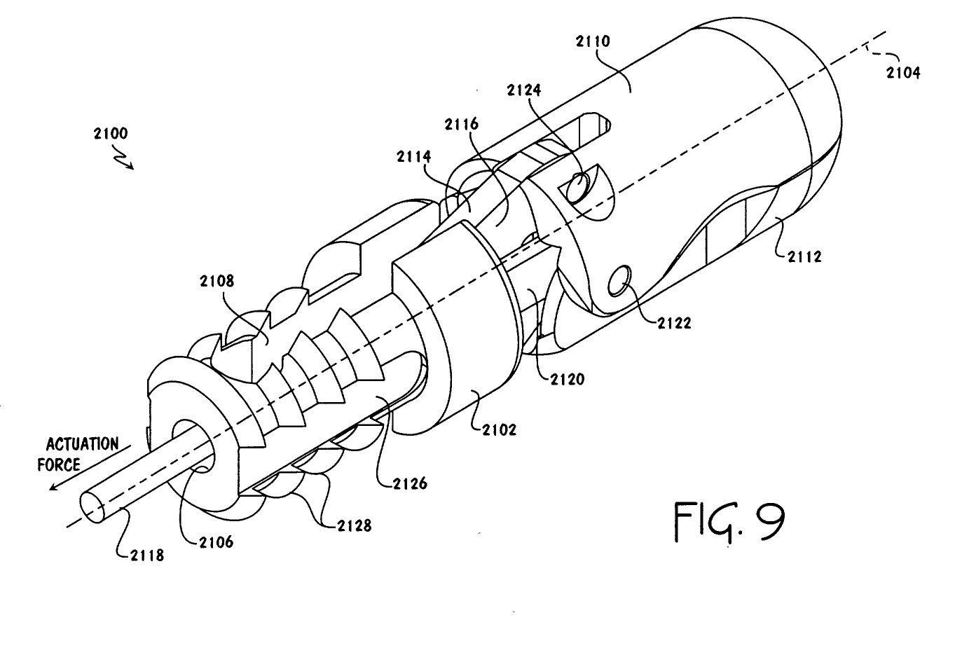

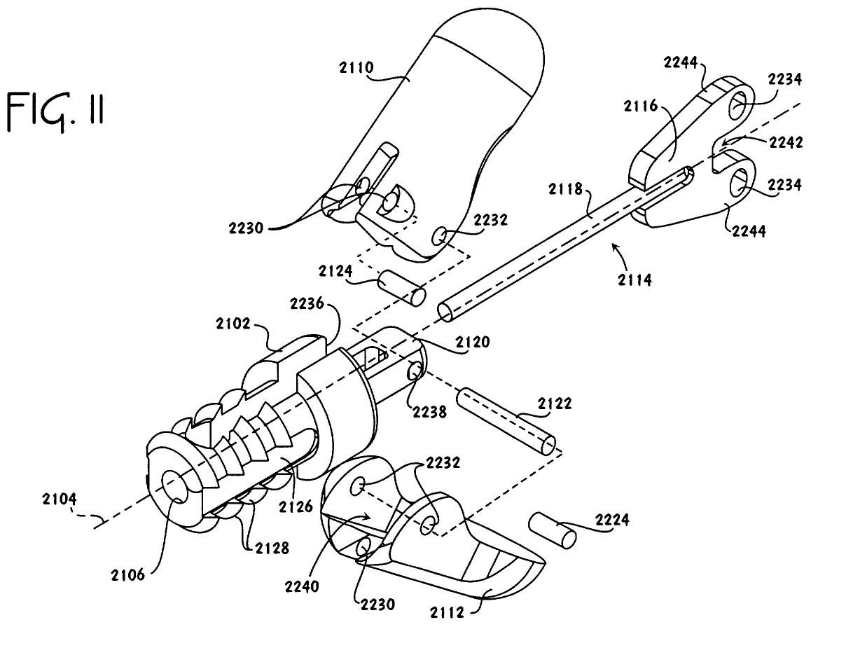

FIGS. 9-14 show a second embodiment of a tissue expansion apparatus 2100 for treating or disrupting a vascular occlusion comprising two tissue expansion members rotating radially around a central hinge assembly. FIG. 11 shows an exploded view of this second embodiment. This second embodiment includes a central internal hinge assembly 2120 rather than the dual hinge assembly arms of the previous embodiment.

Referring to FIG. 9, the tissue expansion apparatus 2100 includes many similar components to the previous embodiment. As before, the tissue expansion apparatus 2100 includes a base section 2102 having a central axis 2104, base aperture 2106, and an actuation channel 2108. In this embodiment, the tissue expansion apparatus 2100 includes a first tissue expansion member 2110, a second tissue expansion member 2112, an actuation assembly 2114 including an actuation plate 2116 and an actuation member 2118, a hinge assembly 2120, a hinge pin 2122 associated with the hinge assembly 2120, coupling pin(s) 2124 associated with each tissue expansion member, a mounting set channel 2126, and retention fins 2128. The central axis 2104 extends through the base section 2102 of the tissue expansion apparatus 2100, as well as through the hinge assembly 2120.

Again referring to FIG. 9, the tissue expansion apparatus 2100 in operation is placed into approximate contact with a vascular occlusion and/or a blood vessel wall to facilitate the disruption of the vascular occlusion. An actuation force, including a proximally exerted linear force, can be applied to the actuation assembly 2114 (i.e., through actuation member 2118), whereupon the actuation force is converted into a spreading or mechanical force (e.g., outward radial force with respect to the hinge assembly 2120). The force can be exerted by the tissue expansion members on the occlusion, between the occlusion and vascular walls, or on the vascular wall itself by the motion of the tissue expansion members (2110, 2112). The spreading or mechanical force being exerted by the tissue expansion members (2110, 2112) may be applied to the vascular occlusion and/or a blood vessel wall so as to tear, fracture or otherwise disrupt, a vascular occlusion located within a section of vasculature without damaging the blood vessel wall, by virtue of the force against the tissue being distributed over the surface of each blunt shaped tissue expansion member (2110, 2112). The continued linear disruption of the vascular occlusion and corresponding advancement of the claimed invention, can create a channel or a passageway of sufficient size for the passage of the apparatus and attached catheter shaft. Once the occlusion has been crossed, a guide-wire or therapeutic catheter can be advanced within the dissected occlusion for elective therapeutic procedures. The advancement of the guide-wire can be along side the catheter shaft or independently after catheter shaft removal.

FIG. 9 shows the tissue expansion apparatus 2100 including a base section 2102 and the tissue expansion members (2110, 2112) coupled to the hinge assembly 2120. The tissue expansion members (2110, 2112) can move radially outward with respect to the hinge assembly 2120. The base section 2102 of the tissue expansion apparatus 2100 includes a base aperture 2106 and an actuation channel 2108, which partially accommodates the actuation member 2118 and the actuation plate 2116.

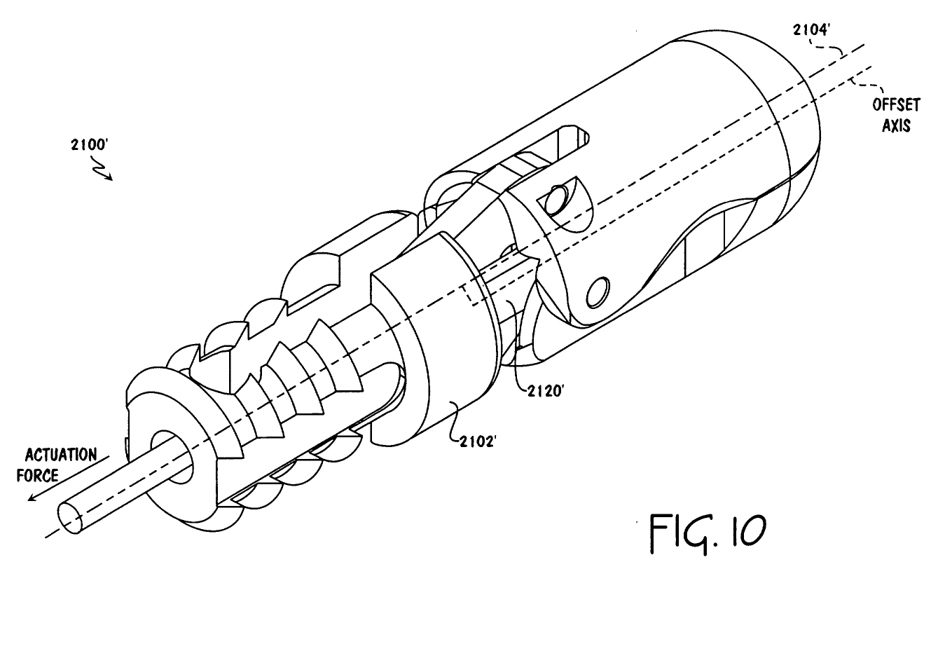

FIG. 10 shows an alternative embodiment where the axis of rotation of the tissue expansion members does not intersect the central axis of the apparatus. The offset axis of rotation results in differing moment arms between the tissue expansion members (2110, 2112) and the hinge assembly 2120. In such an embodiment, each tissue expansion member will convey to the walls of the vessel differing amounts of force as a function of the different moment arm.

As shown in FIG. 11, the tissue expansion members (2110, 2112) include coupling pin apertures 2230 and hinge member apertures 2232. The hinge member apertures 2232 accommodate a hinge pin 2122, which couples the tissue expansion members to the hinge assembly 2120, such that the respective tissue expansion members can move radially with respect to the hinge pin 2122. The coupling pin apertures 2230 accommodate coupling pins 2124, which couple the tissue expansion members to the actuation plate 2116 via the corresponding actuation plate apertures 2234. The coupling between the actuation plate 2116 and the respective tissue expansion members (2110, 2112) allows for the conversion of an actuation force, including a proximally linear actuation force, into the radial motion of the respective tissue expansion members (2110, 2112) around the hinge assembly 2120.

FIG. 11 also shows a tissue expansion apparatus 2100 with a hinge assembly 2120. In this embodiment, the hinge assembly 2120 is “U” shaped, with the ends of the assembly 2120 being affixed to, and/or integrally molded with, the distal face 2236 of the base section 2102. The hinge assembly 2120 includes a hinge assembly aperture 2238 that can accommodate the hinge pin 2122. The hinge pin 2122 engages the hinge member apertures 2232 of the respective tissue expansion member (2110, 2112) and the hinge assembly aperture 2238, coupling the respective tissue expansion member to the hinge assembly 2120. The hinge assembly 2120 provides a central axis around which the respective tissue expansion member (2110, 2112) can move radially outward and inward with respect to the longitudinal axis of the hinge pin 2122. The respective tissue expansion member (2110, 2112) can be coupled to the hinge assembly 2120 via the hinge pin 2122 using standard bonding or coupling techniques allowing for radial movement that are well known to one skilled in the relevant art and as discussed herein.

Each respective tissue expansion member 2110 and 2112 can also have an assembly accommodation area 2240 located within their interior portion to accommodate the hinge assembly 2120. The assembly accommodation area 2240 allows for the unobstructed radial movement of the respective tissue expansion members (2110, 2112) around the hinge pin 2120, such as during opening (engaged) and closing (un-engaged) of the tissue expansion apparatus 2100. Furthermore, the respective tissue expansion members (2110, 2112) can be made from a variety and combination of different materials including, but not limited to, stainless steel, nickel titanium, other shape memory alloys, ceramics, bio-compatible medical plastics.

The respective coupling of the hinge pin 2122 and coupling pin 2124 to the different components of the tissue expansion apparatus 2100 may be implemented in a variety of ways, such as by using physical bonding, adhesive bonding, metal joining methods, or other methods which are well known in the art. For instance, the different components of the tissue expansion apparatus 2100 can be bonded with an epoxy or other appropriate material should they be made of a polymer. Welding, soldering, brazing, or other methods appropriate for bonding metallic components can also be used for bonding the metallic components. In one embodiment, the coupling of the hinge pin 2122 and coupling pin 2124 to the different components of the tissue expansion apparatus 2100 can be implemented through spot welds, so as to retain the hinge pin's 2122 and coupling pin's 2124 functionality (e.g., rotational freedom) within the different components of the tissue expansion apparatus 2100.

The actuation assembly 2114 includes an actuation member 2118, such as an actuation cable, rod or wire for example, coupled to an actuation plate 2116. The actuation member 2118 can be coupled to the actuation plate 2116 by a variety of commonly employed techniques, such as physical bonding, adhesive bonding, and metal joining methods, or other techniques which are well known in the art and commonly employed for bonding or coupling two elements together. The actuation member 2118 can be configured to limit the amount of longitudinal force applied to the tissue expansion members (2110, 2112) of the tissue expansion apparatus 2100 as well as the amount of travel of the tissue expansion members. For instance, a pre-selected amount of force may be established for the actuation member 2118 such that the amount of force applied in excess of the selected limit will merely deform the actuation member 2118 and will not be transmitted to the blood vessel through the tissue expansion members (2110, 2112). As previously described, the use of a nickel-titanium actuation member may self-limit the applied force.

The actuation plate 2116 of the tissue expansion apparatus 2100 includes two actuation plate apertures 2234, a channel section 2242, and outer lobe sections 2244. The two respective apertures 2234 of the actuation plate 2116 are configured to engage the respective coupling pins 2124. The coupling pins 2124 are inserted into the coupling pin apertures 2230 of the respective tissue expansion members (2110, 2112). The tissue expansion members (2110, 2112) are coupled to the actuation assembly 2114 via the respective coupling pins 2124 engaging both the coupling pin apertures 2230 of the tissue expansion members (2110, 2112) and actuation plate apertures 2234 of the actuation plate 2116, such that the proximal actuation force applied to the actuation assembly 2114 (e.g., through the actuation member 2118) is converted into a spreading or rotational motion of the tissue expansion (e.g., radial motion with respect to the hinge assembly 2120). This radial motion transmits the actuation force to the vasculature through the expansion members.

The base section 2102 can also provide an actuation channel 2108 allowing for the movement of the actuation plate 2116 within the actuation channel 2108. In one embodiment, the actuation plate 2116 partially resides and moves within the space defined by the actuation channel 2108. The actuation member 2118, which is coupled to the actuation plate 2116, passes through the base aperture 2106 of the base section 2102 allowing for the unimpeded motion of the actuation assembly. The interior of the base aperture 2106, as well as the exterior of the actuation member 2118 can be coated with Teflon® or a similar material, such that any friction from the movement of sliding the actuation member 2118 through the base aperture 2106 is greatly reduced. Thus, an external actuation force applied to the actuation member 2118 is transferred, through the actuation plate 2116 and coupling pins 2124 to the tissue expansion members (2110, 2112). This transfer causes the radial motion of the tissue expansion members around hinge pin 2122.

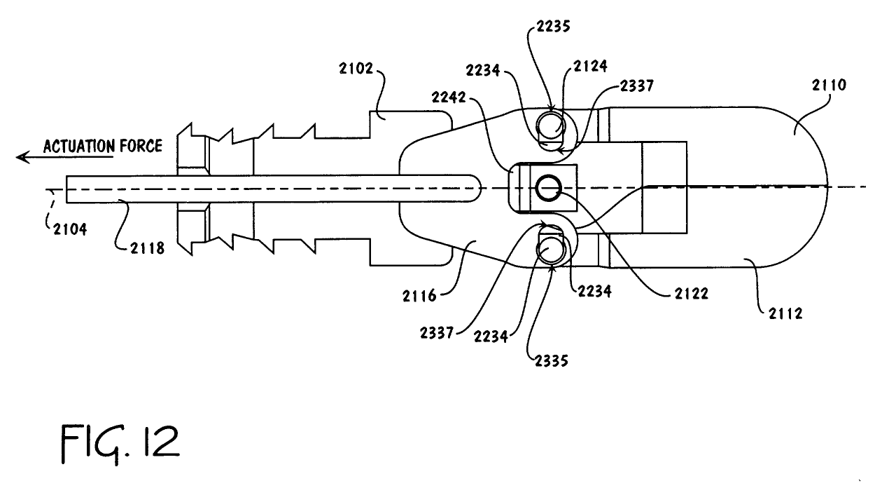

FIG. 12 shows a side cross-sectional view of an embodiment of the tissue expansion apparatus 2100 with the expansion members in a closed or un-engaged position. The tissue expansion members (2110, 2112) are coupled to the hinge assembly 2120 via the hinge pin 2122. As previously described, the hinge pin 2122 provides a central axis about which the expansion members can move radially outwards around the hinge assembly 2120. Additionally, the two apertures 2234 of the actuation plate 2116 can be configured to simultaneously engage the respective coupling pins 2124, which are inserted into the coupling pin apertures 2230 of the tissue expansion members (2110, 2112). The actuation plate 2116, in combination with the coupling pins 2124, transfers the external actuation force applied to the actuation member 2118 to the expansion members, providing for the radial motion of the tissue expansion members (2110, 2112) around the hinge pin 2122. The two actuation plate apertures 2234 of the actuation plate are typically oval shaped, with the longitudinal axis of the oval normal to the central axis 2104 of the assembly, allowing the coupling pins 2124 to change their position as the expansion members rotate. As previously described, the actuation plate apertures 2234 can, however, be of a variety of shapes altering the degree of rotation of each tissue expansion member as well as determining which tissue expansion member rotates first.

When the tissue expansion members (2110, 2112) are in the closed or un-engaged position and the actuation plate apertures, 2234 are oval, the coupling pins 2124 associated with the tissue expansion members are positioned toward the outer sections or edges 2335 of the two apertures 2334 of the actuation plate 2116. As an actuation force is applied to the actuation member 2118, the apertures 2234 associated with the actuation plate 2116 engage the respective coupling pin 2124 resulting in the radial motion of the respective tissue expansion member around the hinge pin 2122. As the tissue expansion members (2110, 2112) rotate around the hinge pin 2122, the coupling pins 2124 reposition themselves toward the inner sections or edges 2337 of the two apertures 2234 of the actuation plate 2116. As described herein the shape of the apertures can be longitudinally and laterally modified to allow non-simultaneous, asymmetric movement of the tissue expansion members. Furthermore, the coupling pins 2124 can be fixed in the actuation plate and translate through oval and similar shaped slots in the expansion members producing like movement of the tissue expansion members.

This embodiment of a tissue expansion 2100 apparatus can allow the tissue expansion members (2110, 2112) of the tissue expansion apparatus 2100 to remain in a closed or un-engaged position until a force is applied to the actuation member 2118. Once a force is applied to the actuation member 2118 in a proximal direction, the expansion members rotate radially outward. The tissue expansion members (2110, 2112) then return either actively or passively to a closed or un-engaged position upon the removal of the actuation force being applied to the actuation member 2118. Active return to a closed position is accomplished by imparting an axially compressive force to the actuation member. The opening mechanism therefore, works in reverse to close the tissue spreading members (2110, 2112). During use of the device, passive closing can also be accomplished by the recoil of vascular tissue onto the tissue spreading members.

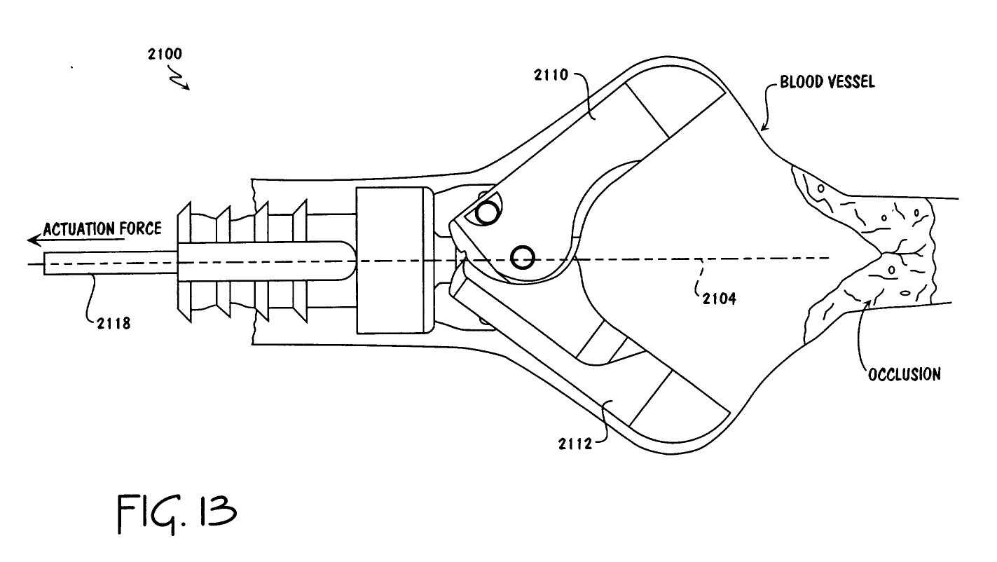

FIG. 13 shows a side view of one embodiment of the tissue expansion apparatus 2100 including two tissue expansion members, with the first tissue expansion member 2110 and second tissue expansion member 2112 in the open or engaged positions. When the tissue expansion members (2110, 2112) are in the open or engaged position, the respective coupling pins 2124 associated with the tissue expansion members are positioned toward the inner sections 2337 of the two apertures 2234 of the actuation plate 2116. From a closed position, as a force is applied, the coupling pins 2124 shift from the outer sections 2335 of the two apertures 2234 toward the inner sections 2337 of the two actuation plate apertures 2234 of the actuation plate 2116. Correspondingly, in response to the actuation force being applied to the actuation member 2118 in the proximal direction, the actuation plate 2116 moves within the actuation channel 2108. The actuation plate 2116, in combination with the coupling pins 2124, transfers the external force to the tissue expansion members (2110, 2112) rotating them around the hinge pin 2122.

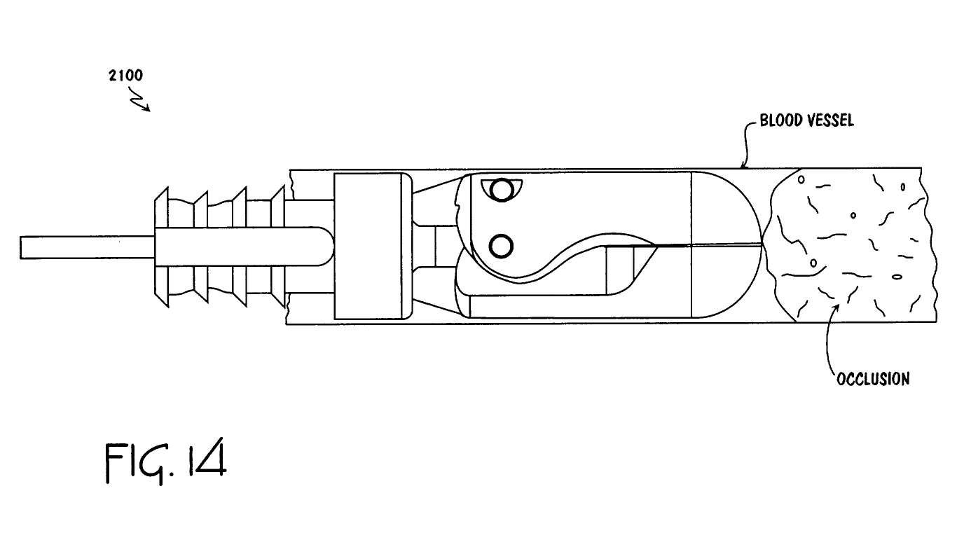

FIG. 14 shows an embodiment of a tissue expansion apparatus 2100 as it is placed adjacent to a vascular occlusion formed within various sections of vasculature. Once the tissue expansion apparatus 2100 is placed into approximate intimate contact with an occlusion, an actuation force is applied to the actuation member 2118, whereupon the respective tissue expansion members (2110, 2112) can move radially outwards with respect to the hinge pin 2122. This radial motion of the tissue expansion members tears, fractures, or otherwise disrupts the body of the occlusion, internally, between the vessel wall and the occlusion or within the vessel wall itself. Continued dissecting and advancement of the tissue spreading assembly establishes a pathway through or around the occlusion, allowing for the placement of guide-wires, intervention devices and catheters across the stenosed vasculature.

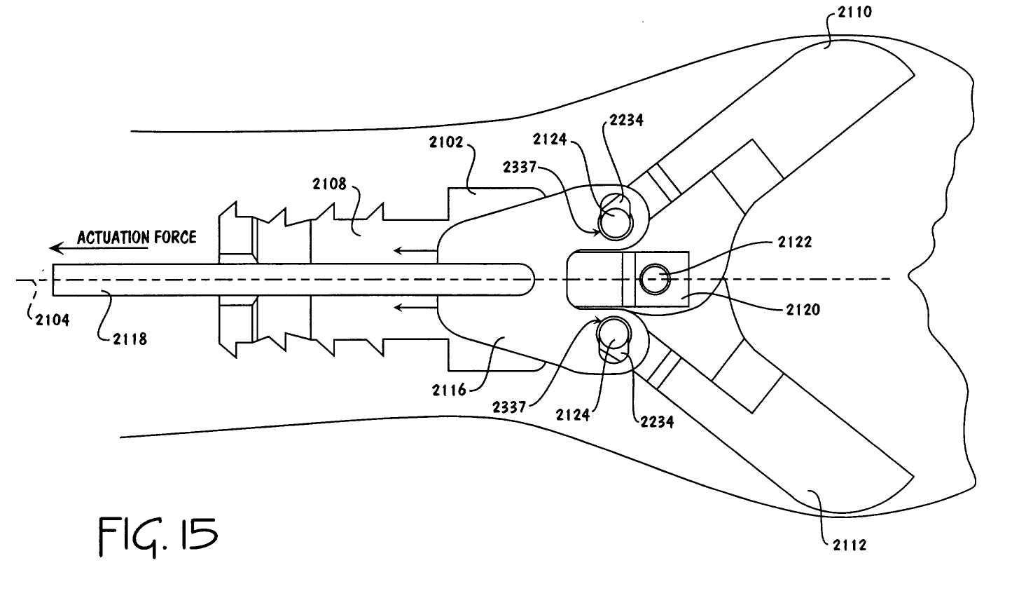

FIG. 15 shows an application of an embodiment of the tissue expansion apparatus 2100 where the outward radial movement of the tissue expansion members (2110, 2112) fractures the body of the occlusion in response to an actuation force being applied to the actuation member 2118. In a further application of this embodiment, the tissue expansion apparatus 2100 can be placed into approximate intimate contact with the occlusion, so that the radial movement of the respective tissue expansion members tears or fractures the body of the occlusion away from the interior lining of an arterial or venous blood vessel. This displacement of the occlusion occurs in response to an actuation force being applied to the actuation member 2118. During this process the blood vessel wall is stretched creating a path substantially between the occlusion and the blood vessel wall.

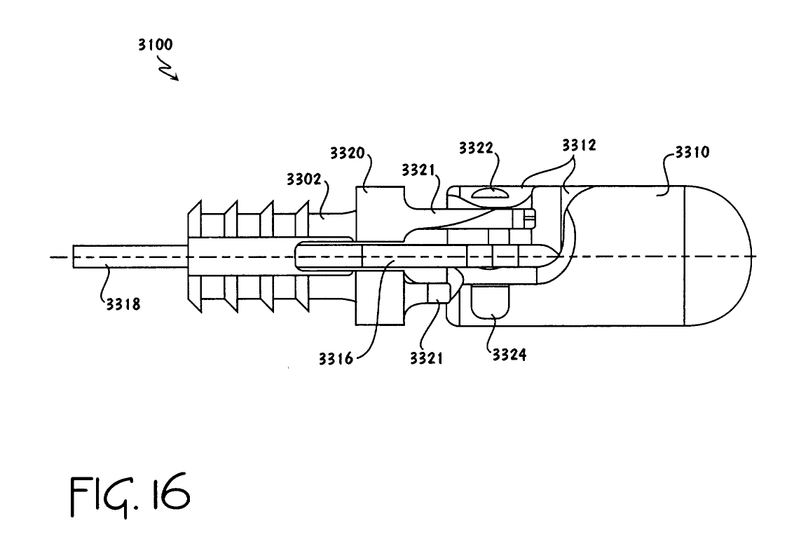

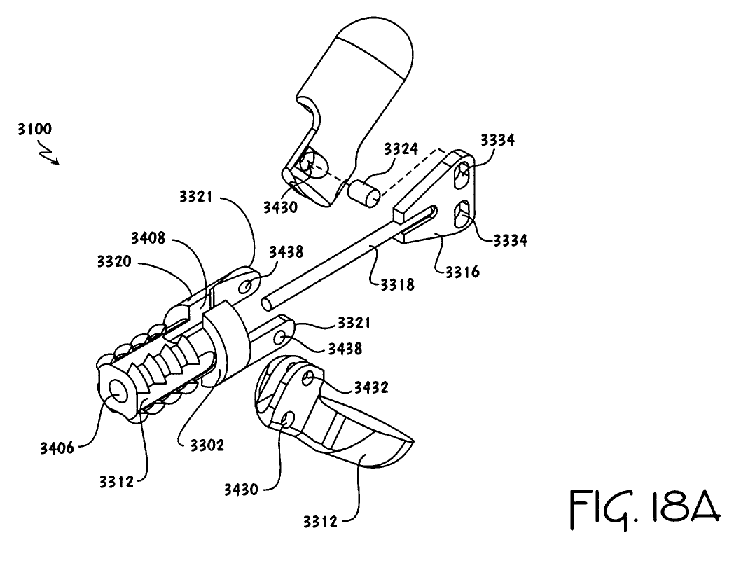

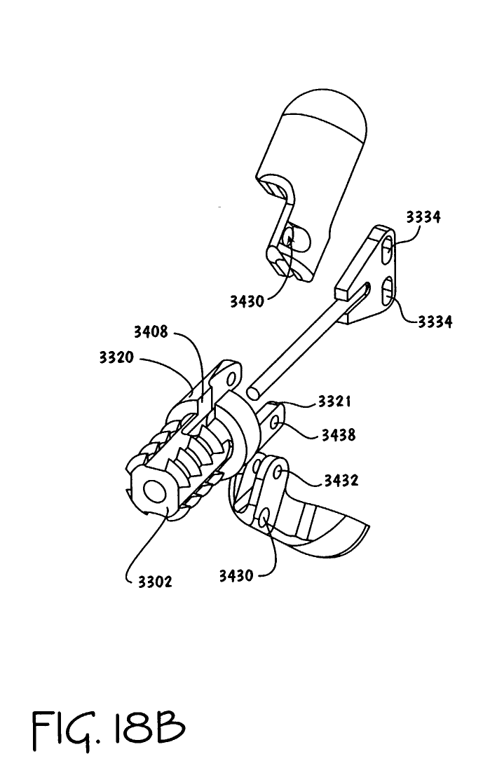

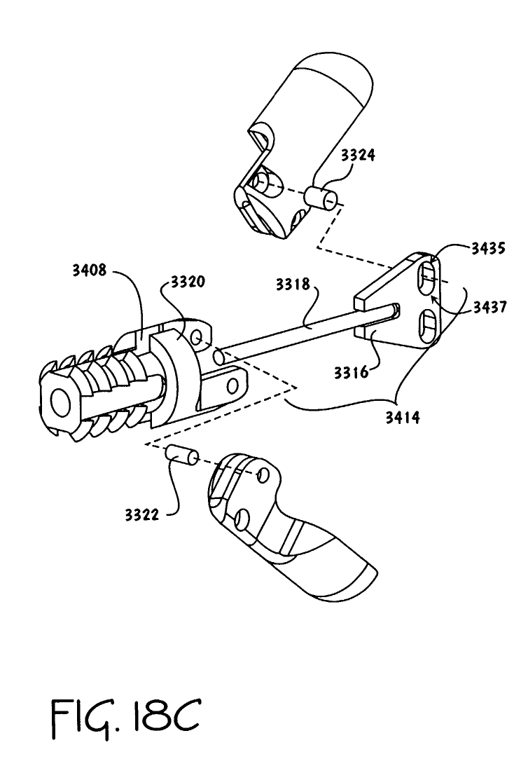

FIGS. 16-22 show another embodiment of a tissue expansion apparatus 3100 for treating or disrupting a vascular occlusion. FIGS. 18A-18C show an exploded view of the same embodiment. This embodiment presents a configuration where each tissue member is coupled to the actuation plate and base section independent of the other tissue expansion member. Additionally, the distance between the hinge pin and coupling pin for each tissue spreading member (3310, 3312) has been maximized, thereby maximizing the moment arm developed between the coupling pin 3324 and the hinge pin 3322 as a proximally exerted force is applied to the actuation member 3318.

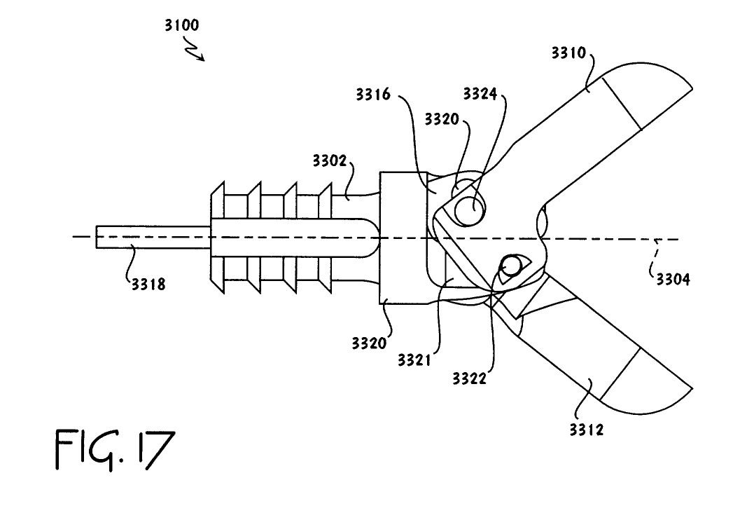

Referring to FIG. 16 and FIG. 17, the tissue expansion apparatus 3100 includes many similar components of the previous embodiments. Like the embodiments previously described, the tissue expansion apparatus 3100 has a base section 3302 having a central axis 3304, base aperture 3406, and an actuation channel 3408. In this embodiment, the tissue expansion apparatus 3100 includes a first tissue expansion member 3310, a second tissue expansion member 3312, an actuation assembly 3314 that includes an actuation plate 3316 and an actuation member 3318, a hinge assembly 3320, hinge pin(s) 3322 and coupling pin(s) 3324 associated with each tissue expansion member, a mounting set channel 3326 (not shown on drawings), and retention fins 3328 (not shown on drawings). The central axis 3304 (not shown on all drawings) extends through the base section 3302 of the tissue expansion apparatus 3100, as well as through the hinge assembly 3320.

In operation, the tissue expansion apparatus 3100 is placed into contact or approximate contact with a vascular occlusion and/or a blood vessel wall to facilitate the disruption of the vascular occlusion. An actuation force, including one exerted linearly in a proximal direction, is applied to the actuation assembly 3314 (i.e., through actuation member 3318), converted into a spreading or mechanical force and motion (e.g., outward radial force and motion with respect to the member's respective hinge pin 3322) and then exerted by the tissue expansion members on the vascular walls. The spreading or mechanical force applied to the vascular occlusion and/or a blood vessel wall tears, fractures or otherwise disrupts, a vascular occlusion without damaging the surrounding blood vessel wall. As described in greater detail for the first embodiment, the continued linear disruption of the vascular occlusion can create a channel or a passageway of sufficient size for the passage of the tissue expansion apparatus and attached catheter shaft to cross the occlusion. A guide-wire or therapeutic catheter can then be advanced within the dissected occlusion for elective therapeutic procedures.

FIGS. 18A-18C further illustrate the tissue expansion apparatus 3100 including a base section 3302 and the tissue expansion members (3310, 3312) coupled to the hinge assembly 3320. The tissue expansion members (3310, 3312) move radially outward with respect to each member's hinge pin 3322. The base section 3302 of the tissue expansion apparatus 3100 includes a base aperture 3406 and an actuation channel 3408, which can partially accommodate the actuation member 3318 and the actuation plate 3316.

The tissue expansion members (3310, 3312) include coupling pin apertures 3330 to accommodate coupling pin(s) 3324, coupling the tissue expansion members to the actuation plate 3316 via the corresponding actuation plate apertures 3334. The coupling between the actuation plate 3316 and the respective tissue expansion members (3310, 3312) allows for the conversion of a proximate linear actuation force, applied to the actuation member 3318 into the radial motion of the respective tissue expansion members (3310, 3312) around the hinge pin 3322.

The base section 3302 of the tissue expansion apparatus 3100 has a hinge assembly 3320 affixed to, or integrally molded with, the distal region of the base section 3302. The distal region includes the distal face and surrounding area of the base section 3302. In this embodiment, the hinge assembly 3320 comprises two hinge assembly arms 3321 extending parallel to the central axis 3304 in the distal direction from the distal face of the base section 3302. The distal face of the base section 3302 is perpendicular to the longitudinal central axis 3304. The two hinge assembly arms 3321 extend from opposite quadrants of the distal face and include a hinge assembly aperture 3438 to accommodate hinge pin(s) 3322. A hinge pin 3322 engages a hinge member apertures 3432 of each respective tissue expansion member (3310, 3312) and the hinge assembly aperture 3438, coupling the respective tissue expansion member to the respective hinge assembly arm 3321. The hinge pin 3322 provides a central axis around which the respective tissue expansion member (3310, 3312) rotate outward and inward relative to the central axis. The respective tissue expansion member (3310, 3312) can be coupled to the respective hinge assembly arm 3321 via the hinge pin 3322 using standard bonding or coupling techniques allowing for their radial movement as previously described.

The actuation plate 3316 contains two actuation plate apertures 3334. The two respective apertures 3334 of the actuation plate 3316 are configured to engage the respective coupling pins 3324 which are inserted into the coupling pin apertures 3430 of the respective tissue expansion members (3310, 3312). The tissue expansion members (3310, 3312) are also coupled to the hinge assembly 3320 via the respective hinge pins 3322 such that a proximal linear actuation of the actuation member 3318 can be converted into a spreading or mechanical force (e.g., radial force with respect to the hinge assembly arm 3321) exerted on the vasculature through the tissue expansion members.

The base section 3302 provides an actuation channel 3408 that allows for movement of the actuation plate 3316. In one embodiment, the actuation plate 3316 partially resides and moves unimpeded within the space defined by the actuation channel 3408. The actuation member 3318, which is coupled to the actuation plate 3316, passes through the base aperture 3406 of the base section 3302. The interior of the base aperture 3406, as well as the exterior of the actuation member 3318 can be coated with Teflon® or a material, such that any friction from the movement of the actuation member 3318 through the base aperture 3406 is minimized.

The two actuation plate apertures 3334 of the actuation plate are, in this embodiment, oval shaped so as to allow the coupling pins 3324 to change their position as the expansion members rotate, similar to the more detailed description provided for the first embodiment. When the tissue expansion members (3310, 3312) are in the closed or un-engaged position, the coupling pins 3324 associated with the tissue expansion members are positioned toward the outer sections or edges 3435 of the two apertures 3334 of the actuation plate 3316. As an actuation force is applied to the actuation member 3318, the apertures 3334 associated with the actuation plate 3316 engage the respective coupling pin 3324 resulting in the radial motion of the respective tissue expansion member around the respective hinge 3322. As the tissue expansion members (3310, 3312) rotate around the hinge pin 3322, the coupling pins 3324 reposition themselves toward the inner sections or edges 3437 of the two apertures 3334 of the actuation plate 3316. As described herein, and as explained in greater detail elsewhere herein, the shape of the apertures 3334 can be varied both longitudinally and laterally to alter the initiation of movement of the tissue expansion member (3310, 3312) as well as altering the degree of rotation of each tissue expansion member. As also described earlier, the coupling pins may be made an integral part of the actuation plate 3316, and travel within slots 3430 of the tissue expansion members.

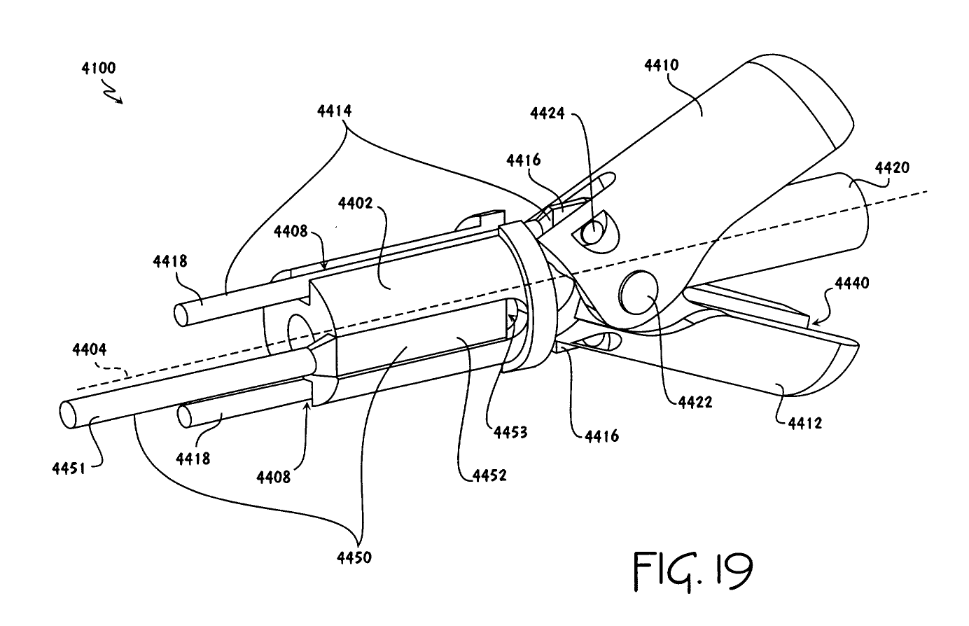

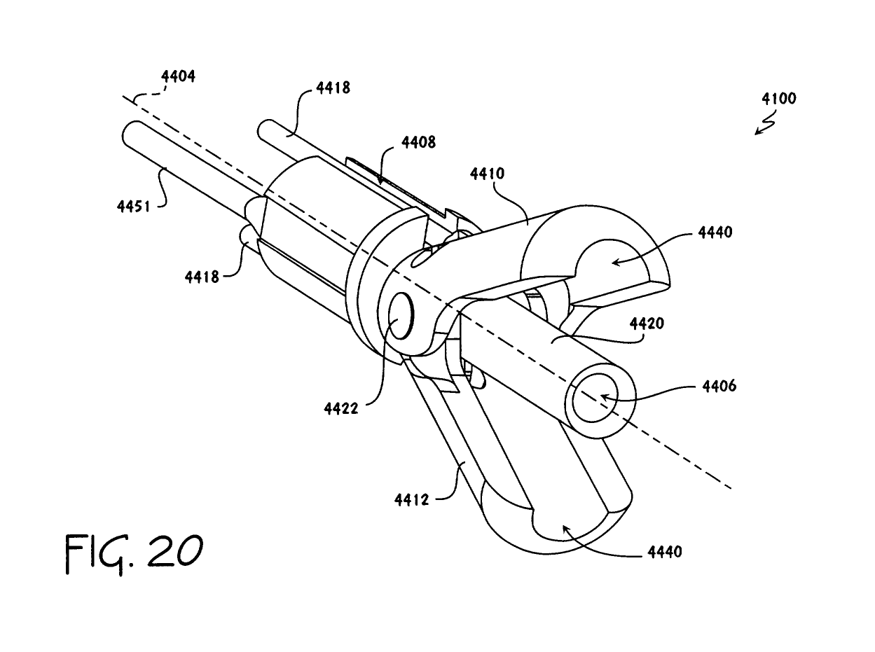

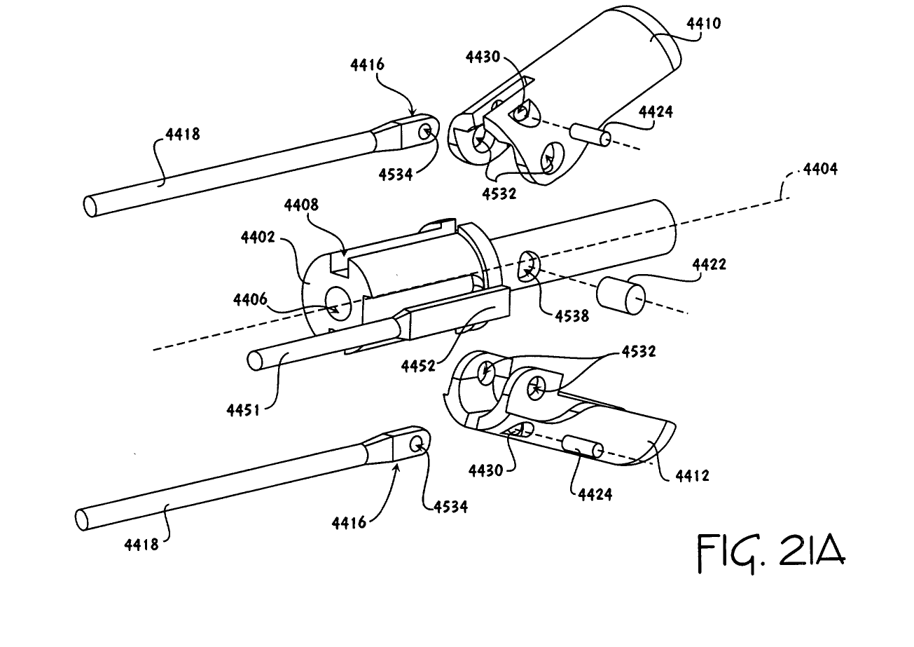

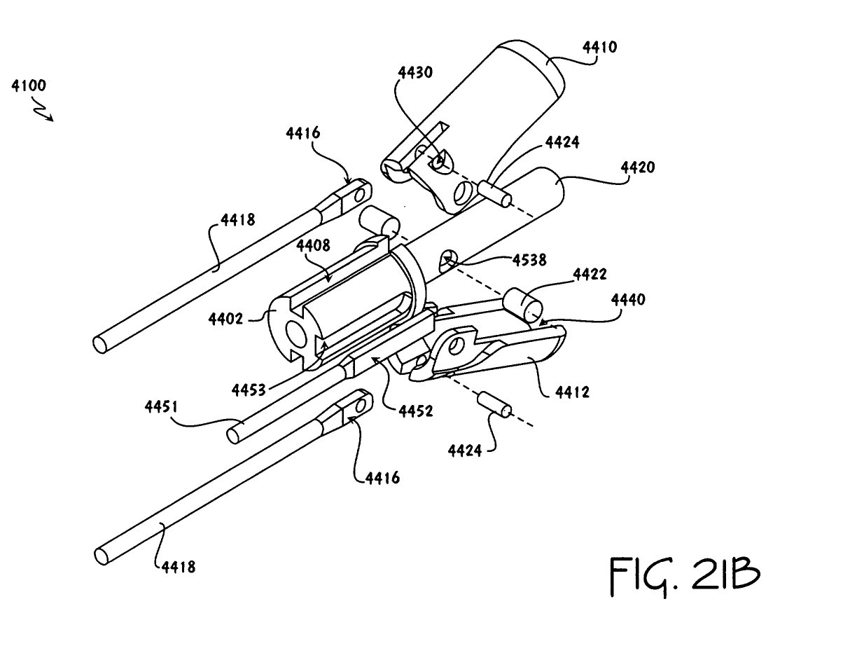

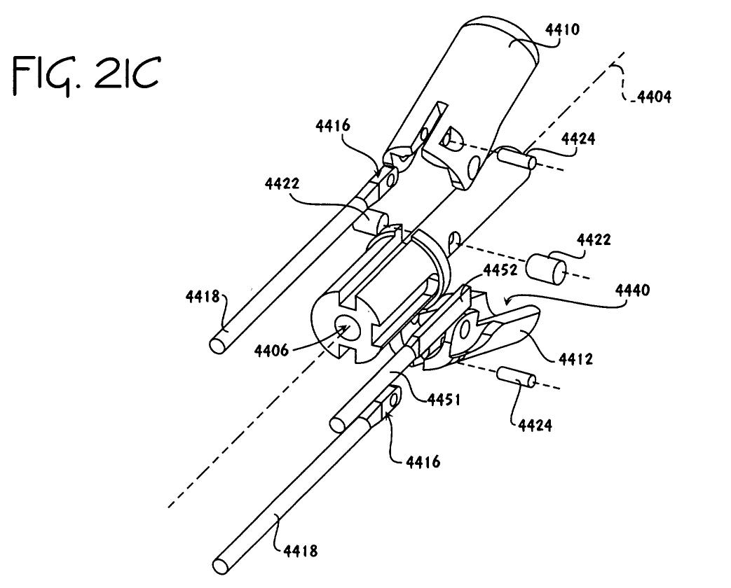

Yet another embodiment of a tissue expansion device is shown in FIGS. 19-24. This embodiment shows the presence of a lumen that traverses the longitudinal length of the apparatus. The lumen can allow the catheter to be tracked along a guide-wire which has been introduced up to the proximal site of the stenosis, i.e. to deliver the catheter to the proximal site of the lesion. Once at the lesion, the catheter will have reached the end of the guide wire. The catheter is then actuated in the same manner as described in previous embodiments herein. As the catheter dissects a tract through or around the occlusion, the guide wire may be advanced at any time if softer areas of the occlusion have been reached which do not require blunt dissection. Therefore, the device is incrementally advanced through the lesion, either following the dissection tract just produced by the tissue spreading members through difficult occluded areas, or following the guide wire after it has been advanced through easier areas of the occlusion. The assembly is advanced in this manner until it reaches the terminal end of the occlusion, and accesses the vessel lumen distal to the occlusion. After the tissue expansion device has reached the terminal side of the occlusion, a guide-wire can be extended into the clear section of the blood vessel and remain in position as the tissue expansion device is removed. The wire can then be used to guide other therapeutic and inter-vascular devices to allow such procedures as DCA, balloon angioplasty, stent delivery and the like. Furthermore, this embodiment includes steering members such that a user can maneuver the tissue expansion device within the vasculature. The steering members are useful when navigating the device through difficult tortuosity of the occlusion, since the assembly has limited ability to track over the guide wire, which typically remains retracted inside the device. FIGS. 19 and 20 illustrate differing views of a tissue expansion apparatus 4100 for treating or disrupting a vascular occlusion. FIGS. 21A-21C show an exploded view of the same embodiment.

Referring to FIG. 19 and FIG. 20, the tissue expansion apparatus 4100 includes a base section 4402 having a central axis 4404, a lumen 4406, two actuation channels 4408, and a steering channel 4453. In this embodiment, the tissue expansion apparatus 4100 further includes a first tissue expansion member 4410, a second tissue expansion member 4412, two actuation assemblies that each include an actuation plate 4416 and an actuation member 4418, a distal lumen/hinge assembly 4420, a hinge pin 4422 associated with the base section 4402, and coupling pin(s) 4424 associated with each tissue expansion member. Other embodiments may contain additional tissue expansion members.

This embodiment also contains a steering assembly 4450 comprising a steering member 4451 and a steering plate 4452. A steering channel 4453 accommodates the steering plate 4452 of the steering assembly 4450 such that the steering plate 4452 can be coupled to the base section 4402. Furthermore, the steering member 4451 is coupled to the steering plate 4452. The coupling of the steering plate 4452 to the base section 4402 and the coupling of the steering member 4451 to the steering plate 4452 can be accomplished by a variety of commonly employed techniques, such as physical bonding, adhesive bonding, and metal joining methods, or other techniques which are well known in the art and commonly employed for bonding or coupling two elements together.

The central axis 4404 extends through the base section 4402 of the tissue expansion apparatus 4100, as well as through the lumen 4406 which transverses the entire apparatus. The lumen 4406 begins at the proximal end of the base 4402 and continues through the tissue expansion apparatus 4100 to the distal end of the distal lumen/hinge assembly 4420. The lumen can accommodate the presence of a guide-wire, catheter, or other intervention device.

In operation, the tissue expansion apparatus 4100 can be placed into contact or approximate contact with a vascular occlusion and/or a blood vessel wall to initiate a dissected pathway across the vascular occlusion. The apparatus may also be used to dissect a pathway between the vascular occlusion and the vessel wall, or within the vessel wall itself. This placement can be facilitated or controlled by the steering assembly 4450. The application of a linear force in the proximal or distal direction of the steering member, while not advancing the catheter, can displace the apparatus laterally to facilitate the proper positioning of the apparatus relative to the occlusion. In other embodiments the apparatus may comprise more than one steering assembly.

An actuation force can be applied independently to either actuation assembly 4414 (i.e., through actuation member 4418), whereupon a proximal linear actuation force is converted into a spreading motion (e.g., outward radial motion with respect to the hinge assembly 4420) that is conveyed to the vascular walls via the tissue expansion members. The continued spreading or mechanical force being exerted independently by the respective tissue expansion members (4410, 4412) can be applied to the vascular occlusion repetitively so as to tear, fracture, dissect or otherwise disrupt, the vascular occlusion without damaging the blood vessel.

FIG. 20 further illustrates the tissue expansion apparatus 4100 including a base section 4402 and the tissue expansion members (4410, 4412) coupled to the hinge assembly 4420. The tissue expansion members (4410, 4412) move radially outward with respect to their corresponding hinge pin 4422 while preserving the lumen 4406 that transgresses the entire apparatus. The base section 4402 of the tissue expansion apparatus 4100 also includes two actuation channels 4408 that accommodate the actuation members 4418 and the actuation plate 4416.

As shown in FIGS. 21A-21C, the tissue expansion members (4410, 4412) include coupling pin apertures 4530 (4430 in FIG. 21) and hinge member apertures 4532. The hinge member apertures 4532 accommodate a hinge pin 4422, which couples the tissue expansion members to the hinge assembly/distal lumen 4420, such that the respective tissue expansion members can move radially with respect to the individual hinge pin 4422. The coupling pin apertures 4530 accommodate coupling pins 4424, which couple the tissue expansion members to their respective actuation plate 4416 via the corresponding actuation plate apertures 4534. The coupling between the actuation plate 4416 and the respective tissue expansion members (4410, 4412) allows for the conversion of a actuation force, applied to the actuation members 4418, into independent radial motion of the respective tissue expansion members (4410, 4412) around the hinge pin 4422.

FIGS. 21A-21C also show the base section 4402 of the tissue expansion apparatus 4100 that is affixed to, and/or integrally molded with a hinge assembly/distal lumen 4420. The hinge assembly/distal lumen 4420 is coupled to the distal region of the base section 4402 which includes the distal face and surrounding area. In one embodiment, the hinge assembly/distal lumen 4420 comprises a cylindrical tube extending longitudinally and parallel to the central axis 4404. The hinge assembly distal lumen 4420 also accommodates the lumen 4406 aligned with the central axis of and traversing through the apparatus 4410. The hinge assembly 4420 includes two hinge assembly apertures 4538 to accommodate a hinge pin 4422 for each side of the tissue expansion members (4410, 4412). The hinge pins 4422 engage the hinge member apertures 4532 of the respective tissue expansion member (4410, 4412) at the hinge assembly apertures 4538, coupling the respective tissue expansion member to the hinge assembly 4420. The hinge pins 4422 are perpendicular to the central axis 4404 providing an axis by which the tissue expansion members (4410, 4412) can move radially outward and inward. The hinge pins 4422 are positioned to couple the respective tissue expansion member to the hinge assembly/distal lumen 4420 but do not protrude into the lumen traversing the tissue expansion device, maintaining this lumen for the passage of a guide wire or other devices. The respective tissue expansion member (4410, 4412) can be coupled to the hinge assembly/distal lumen 4420 via a hinge pin 4422 using standard bonding or coupling techniques allowing for their radial movement as described herein.

In this embodiment, the two actuation assemblies 4414 each comprise an actuation member 4418 coupled to an actuation plate 4416. The actuation member 4418 can be coupled to the actuation plate 4416 by a variety of commonly employed techniques, as previously described. As each tissue expansion member (4410, 4412) is coupled to an independent actuation assembly 4414, the movement of the tissue expansion members can be independent of one another. In another embodiment, the actuation member 4418 may be configured to limit the amount of longitudinal force applied to the tissue expansion members (4410, 4412) or limit the range of motion of the tissue expansion members (4410, 4412). General embodiments are discussed more specifically herein.

Each actuation plate 4416 includes a single actuation plate aperture 4534. The actuation plate aperture 4534 of the actuation plate 4416 is configured to engage the respective coupling pin 4424 which is inserted into the coupling pin apertures 4430 of the respective tissue expansion member. The tissue expansion members (4410, 4412) are coupled to the actuation assemblies 4414 via their respective coupling pins 4424 and actuation plates 4416, such that the actuation force applied to the actuation assembly 4414 (e.g., through the actuation member 4418) conveys a spreading or mechanical force (e.g., radial force with respect to the central axis) to the vasculature members.

The base section 4402 also provides actuation channels 4408 allowing for the movement of the actuation plates 4416 and actuation members 4418. In the embodiment shown, the actuation member 4418 resides and moves within the space defined by the actuation channel 4408. The interior of the actuation channels 4408, as well as the exterior of the actuation members 4418, can be coated with Teflon® or a similar material, such that any friction from the movement of the actuation member 4418 through the actuation channel 4408 is minimized. As a result, any actuation force applied to the actuation member 4418 is transferred to the actuation plate 4416 and coupling pins 4424 ultimately causing the radial motion of the respective tissue expansion members (4410, 4412). Furthermore, the depth of the actuation channel 4408 is sufficient such that the lateral displacement of the actuation member 4418 as result of the radial movement of the tissue expansion members (4410, 4412) does not cause the actuation member's 4418 motion to be restricted.

In an embodiment illustrated in FIGS. 21A-21C, each tissue expansion member includes a member channel 4440, allowing each tissue expansion member to accommodate the exterior contour of the hinge assembly/distal lumen 4420 which in turn houses the lumen 4406. Furthermore, each actuation plate aperture 4534 of the actuation plate can be oval or similarly shaped allowing the coupling pins 4424 to change their position as the expansion members rotate. As an alternate embodiment, the coupling pin 4424 can be affixed to the actuation plate 4416 and couple within the aperture 4430. Additionally, the shape of the aperture 4430 may be varied to allow the coupling pin 4424 to change position within it as the tissue expansion members (4410, 4412) rotate through their opened and closed positions.

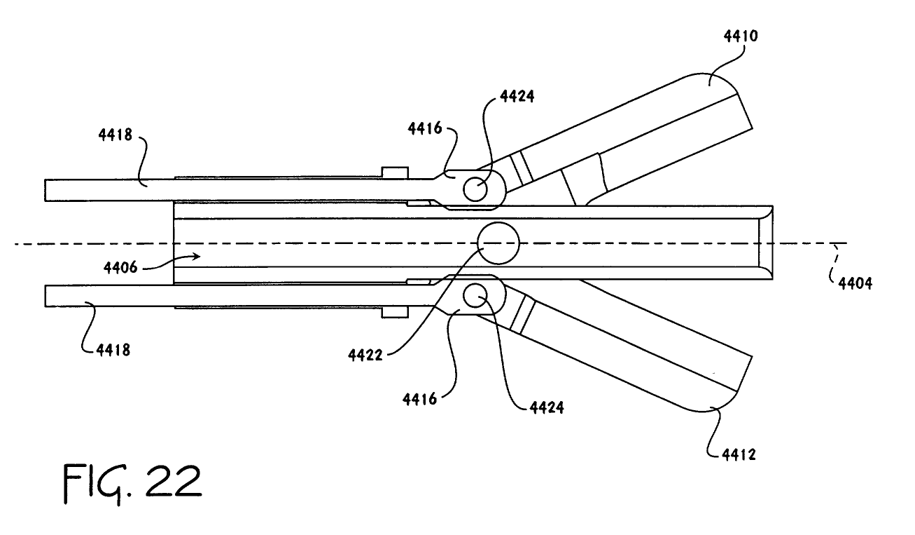

FIG. 22 shows a side cross-sectional view of this embodiment of a tissue expansion apparatus 4100. The first tissue expansion member 4410 and second tissue expansion member 4412 are in the open or engaged positions. When the tissue expansion members (4410, 4412) are in the open or engaged position, the respective coupling pin 4424 associated with the tissue expansion member is positioned toward the inner sections 4537 (not shown on drawings) of the apertures 4534 of the actuation plate 4416. In response to an actuation force being applied to the actuation member 4418 in a proximal direction, the coupling pin 4424 shifts from the inner section of the aperture to the outer section of the aperture transferring a proximal linear force to the respective tissue expansion members (4410, 4412) independently, causing the tissue expansion members to close around the hinge pin 4422.

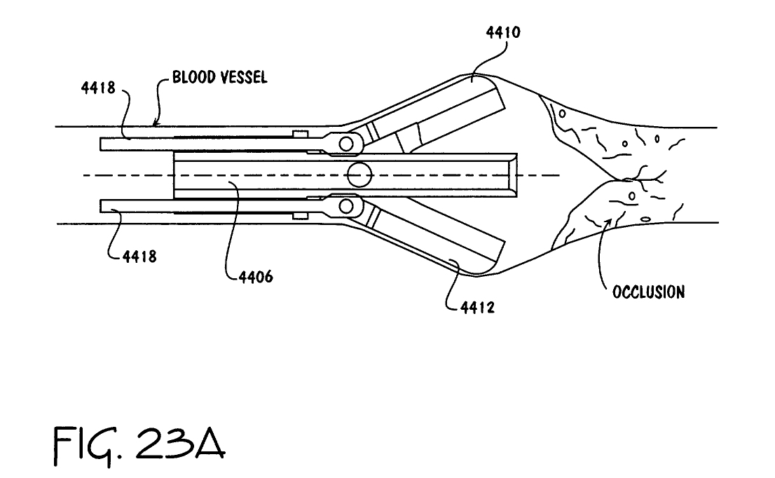

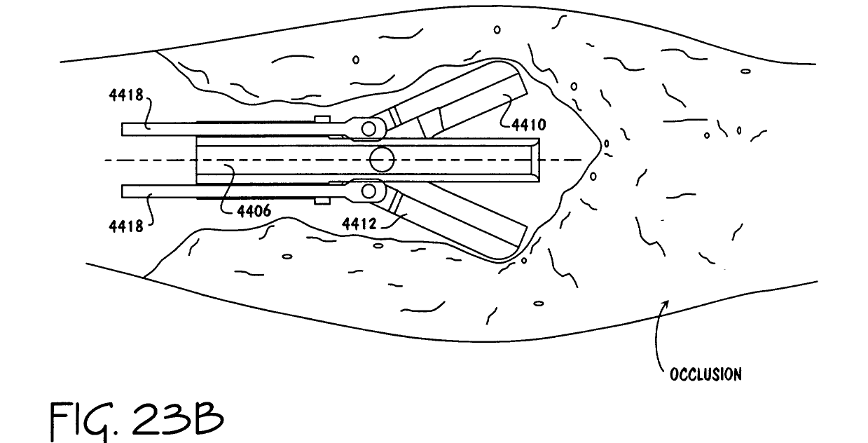

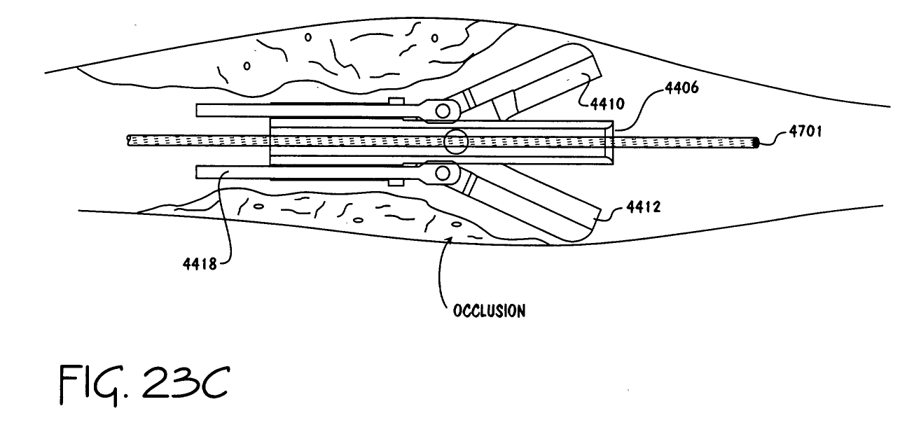

FIGS. 23A-23C show an embodiment of an apparatus for dissecting a vascular occlusion as it traverses a chronic total occlusion. As the device is placed in contact with the occlusion, the tissue expansion members expand the vasculature such that a fissure or tear is created within the occlusion. This fissure may also take the form of the occlusion becoming separated from one or more sections of the blood vessel wall such that the tissue expansion apparatus 4100 can advance within the corresponding space. The dissection may also occur within the vessel wall itself. Through the continued and repetitive expansion of the tissue expansion members (4410, 4412), the apparatus 4100, which is attached to a catheter shaft, can advance through or around the occlusion.

Upon reaching the terminal (distal) side of the occlusion, a pathway has been established from the distal end to the proximal end of the occlusion. With the catheter and apparatus on the distal side of the occlusion, a guide-wire 4701 or similar device, can be introduced through the lumen 4406 such that the distal end of the guide-wire 4701 is distal to the occlusion. After the guide-wire 4701 has been introduced, the tissue expansion apparatus 4100 can be withdrawn leaving the guide-wire 4701 in place to guide devices capable of DCA or other therapeutic procedures.

The actuation assemblies described herein are coupled to a catheter wire or cable which is used to convey the linearly exerted force from an actuator to the tissue expansion apparatus. In this and other embodiments the thickness of the wire can vary over its length. The wire can be tapered such that the distal end of the wire, or where it attaches to the actuation assembly, can be very small. Traveling proximally, the wire's diameter can increase until it reaches its final size.

The actuation wire attached to the actuation assembly possesses sufficient tensile strength to actuate the various linkages in the device. Thus the wire is strong enough to open the tissue expansion members as well as possesses sufficient rigidity to actuate the linkage to close the tissue expansion members. As the wire is an integral component of the catheter, it directly affects the catheter's flexibility. By reducing the thickness of the wire at the distal end, the flexibility of the catheter can be increased. This allows the catheter and device to traverse the tortuous nature of coronary and similar vasculature.

The realized stress of the wire can remain within design limitations even as the diameter of the wire is reduced. As the wire traverses the vasculature of the body, the tensile stress imposed on any one cross-sectional area of the wire changes. The longitudinal stress in the wire is at a maximum at the proximal actuator and decreases to a minimum at the distal apparatus due to frictional losses as the wire comes into contact with the internal catheter lumen which houses the wire. The greatest tortuosity of the catheter, and hence of the wire occurs at the distal portion of the catheter, and typically the distal 8 to 20 cm. Thus the frictional losses cumulatively increase at further distal positions along the catheter, and the proximal input force is incrementally reduced at further distal positions along the catheter. Typically only 10-20% of the actual proximal force input at the actuator is experienced by the distal actuating assembly. The wire itself, however, should remain capable of withstanding the stress imposed during an actuation process without any frictional losses.

Tapering the wire such that its diameter is minimized at the distal end and increases in the proximal direction can improve flexibility of the catheter as it attempts to traverse the vascularture near an occlusion. As well, further distal portions of the wire are required to withstand decreasingly less force, consistent with the frictional losses described previously. The distal diameter can be in the range of 0.0102 to 0.0254 cm, preferably within the range of 0.014 to 0.018 cm, without detrimentally impacting the functionality of the device. Likewise the proximal end of the taper can be 0.0178 to 0.0508 cm, preferably within the range of 0.018 to 0.023 cm. The taper can take place over a wire length from 5 cm to 63 cm, preferably 30 cm. The actual diameter of the wire is dependent on the device being actuated and the application of the catheter system. The tapering can be accomplished by a linear, exponential, serial section or any other mathematical relationship meeting the needs of the catheter system. Furthermore, the taper can be interrupted by straight wire segments.

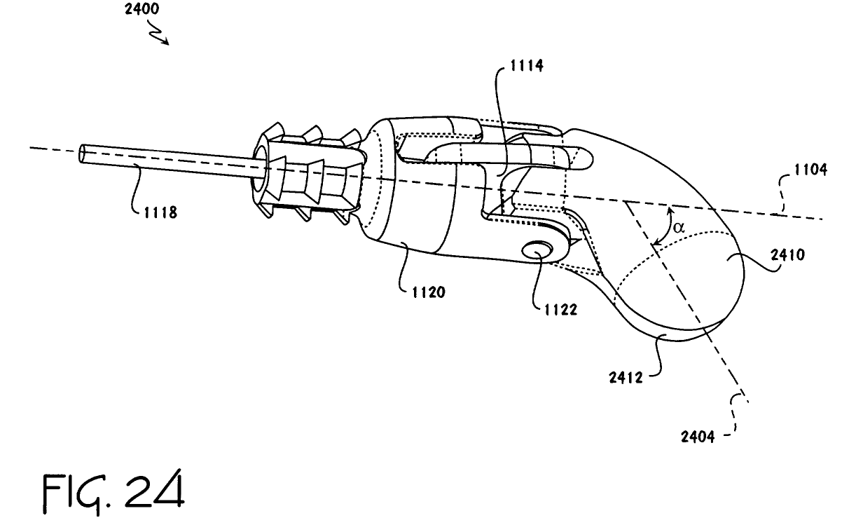

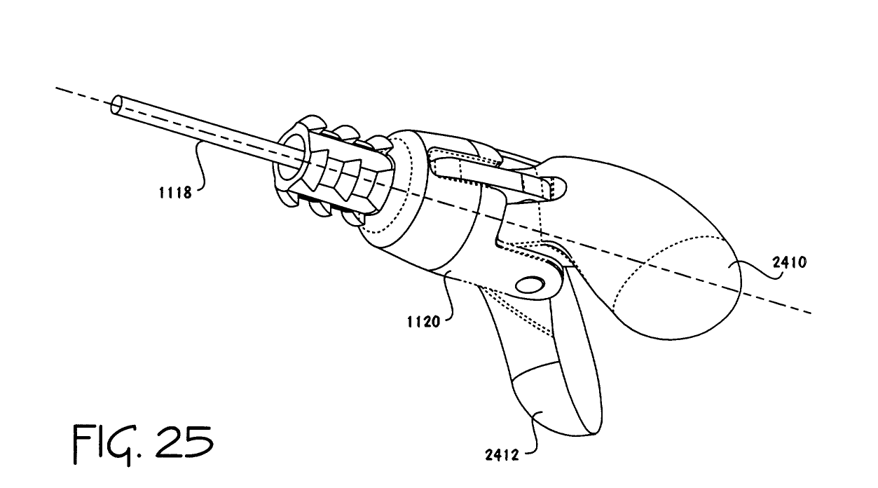

FIG. 24 shows an alternative embodiment of an apparatus 2400 for micro-dissection of vascular occlusions using a tissue expansion member shape that is offset from the central axis 1104. In this embodiment, the individual tissue expansion member's (2410, 2412) longitudinal axis 2404, which is orthogonal to the distal end of the apparatus, is offset α degrees from the central axis 1104. The amount of angular displacement of the tissue expansion member's longitudinal axis 2404 from the central axis 1404 can vary depending on the applicable use of the apparatus. The operation of the micro-dissection apparatus 2400 and functionality of the components associated with the movement of the tissue expansion members (2410, 2412) remain consistent with other embodiments described herein. For clarification, FIG. 25 shows the micro-dissection apparatus 2400 of FIG. 24 in an engaged or open condition.

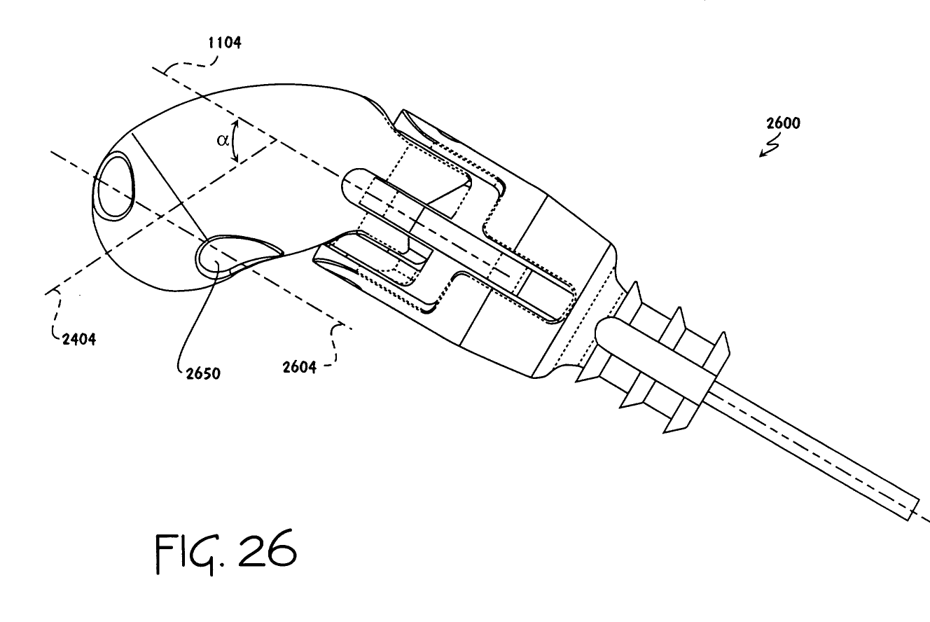

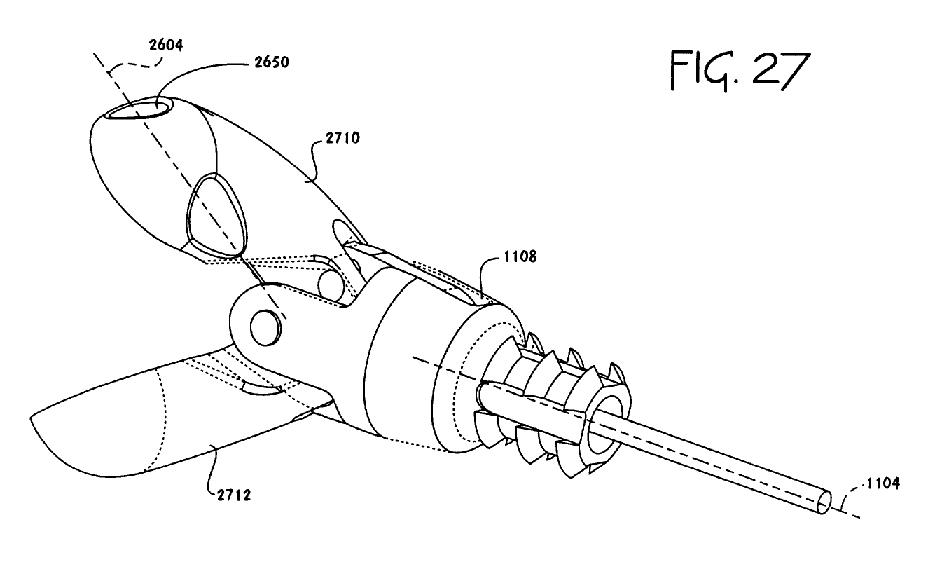

FIG. 26 shows an embodiment of an apparatus for micro-dissection of vascular occlusions 2600 that includes an offset tissue expansion member configured to include a lumen. FIG. 27 shows the micro-dissection apparatus of FIG. 26 in the engage or open position. In this embodiment, a lumen 2650 can traverse a tissue expansion member such that the plane defined by the arc of the lumen's axis 2604 as the tissue expansion member rotates around the hinge assembly is parallel with a plane defined by the actuation channel 1108. The lumen can be of sufficient diameter to accept a guide wire or other cylindrical device of lesser or equal diameter possessing sufficient flexibility so not to impede the motion of the tissue expansion member. The lumen 2650 is typically displaced laterally from the central axis 1104 such that a guide wire, or other device occupying the lumen, will not come into contact or impede the opening and closing of the tissue expansion members. This lumen may be utilized to facilitate tracking the assembly over a guide wire to the target occlusion site.

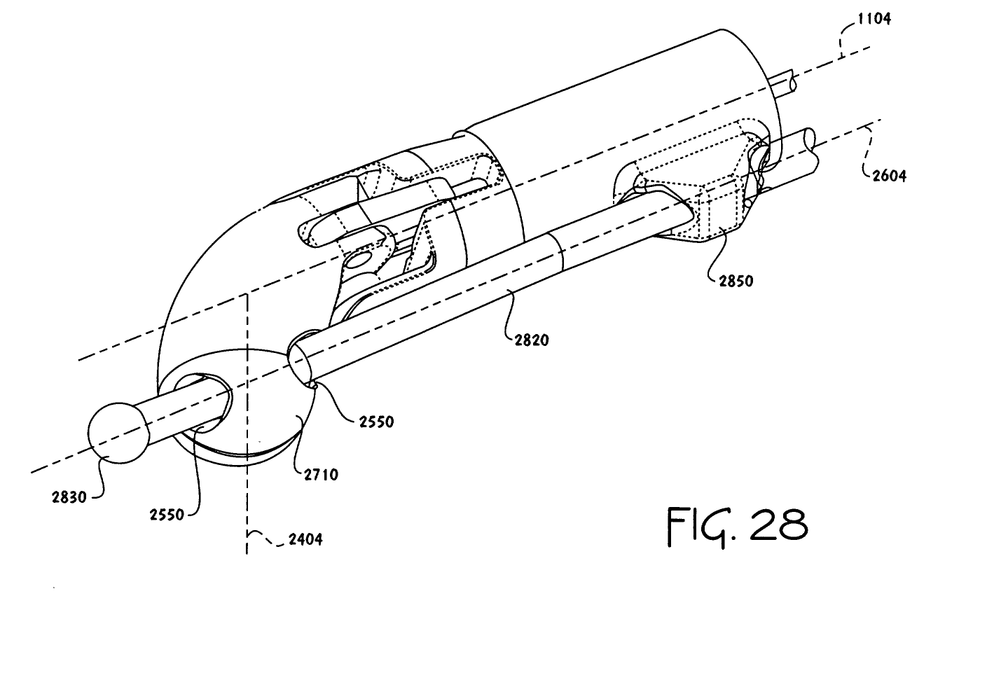

FIG. 28 further illustrates the apparatus embodied in FIG. 26 and FIG. 27 by showing the presence of a guide wire 2820 traversing the lumen 2550. The guide wire 2820 can include a spherical or similarly shaped attachment 2830 to the distal end of the guide wire 2820 that can prevent the end of the guide wire 2820 from becoming disengaged from the tissue expansion member 2710. The attachment 2830 can be coupled to the distal end of the guide wire 2820 using methods known to one skilled in the relevant art. The guide wire 2820 can also be directed along an axis parallel with the plane defined by the actuation channel 1108 by an alignment guide 2850. The alignment guide 2850 can be located proximal to the apparatus on the catheter housing or base section so as to aid in the guide wire's 2820 freedom of movement during the opening and closing of the tissue expansion members (2710, 2712). The flexibility and elasticity of the guide wire is sufficient to allow the complete range of motion of the micro-dissection apparatus. The location of the alignment guide 2850 relative to the tissue expansion member lumen 2550, as well as the channel length of the lumen 2550, can be adjusted to prevent buckling, binding or other mechanical restrictions of the wire within the lumen 2550 or channel of the alignment guide 2850.

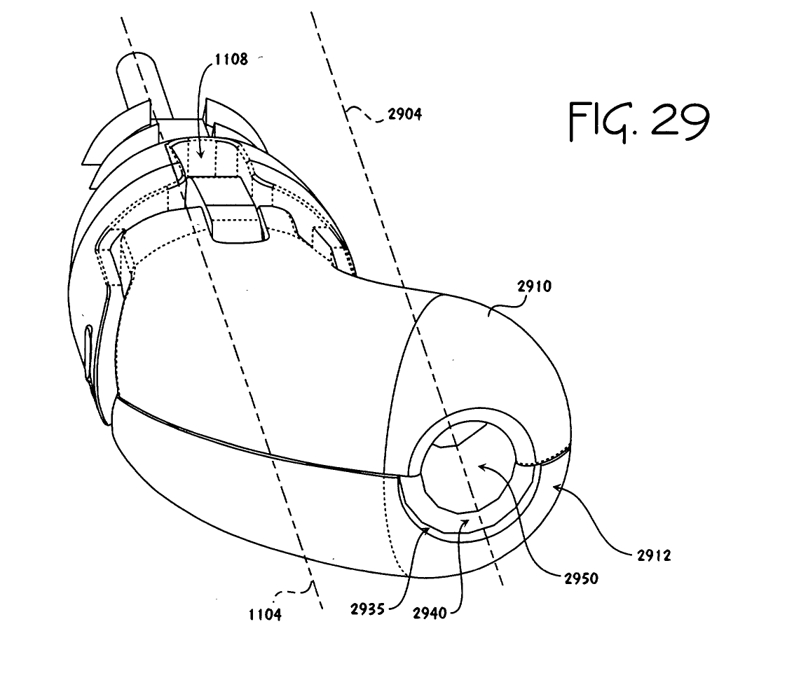

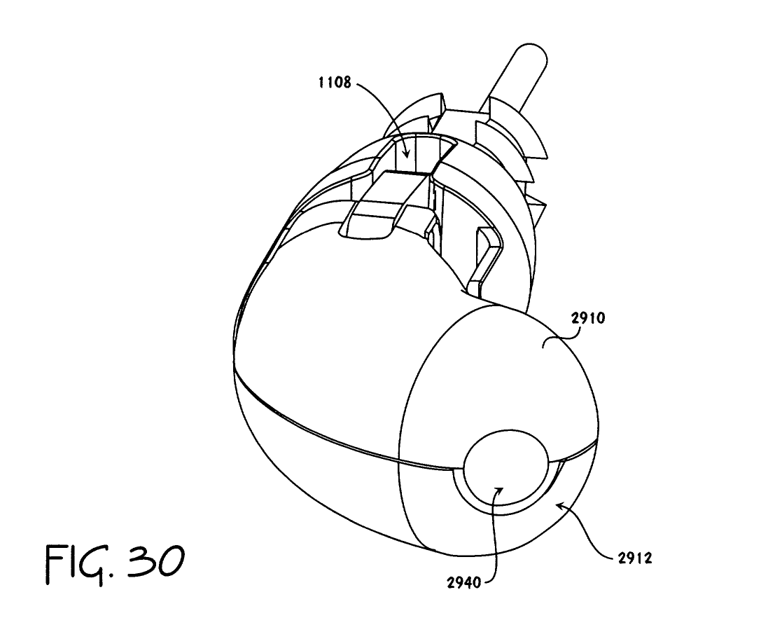

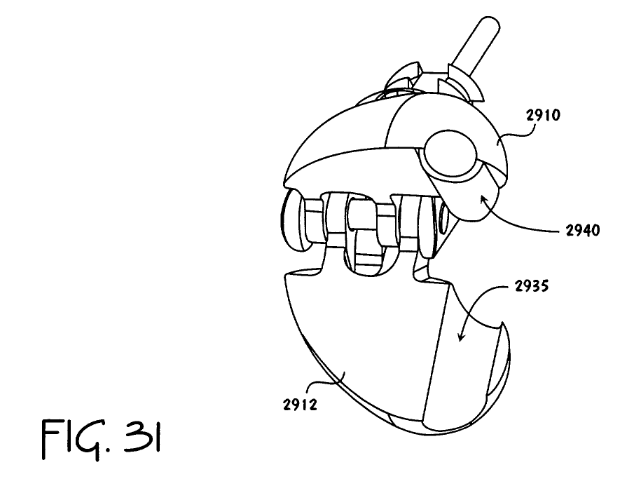

FIGS. 29-31 show an alternative embodiment of an apparatus for micro-dissection of vascular occlusion that includes a lumen traversing a tissue expansion member along an axis that is parallel to the central axis 1104. The lumen, in this embodiment, is housed in a conduit 2940 recessed into one tissue expansion member 2910 and protruding into the other tissue expansion member 2912. The lumen is bisected by the plane perpendicular to the plane defined by the actuation channel 1108. The lower tissue expansion member 2912 accordingly includes a recessed area 2935 on its interior surface to accommodate the conduit 2940 from the upper tissue expansion member 2910. The size and shape of the lumen can vary as described herein so as to not prevent the operational and functional use of the micro-dissection apparatus.

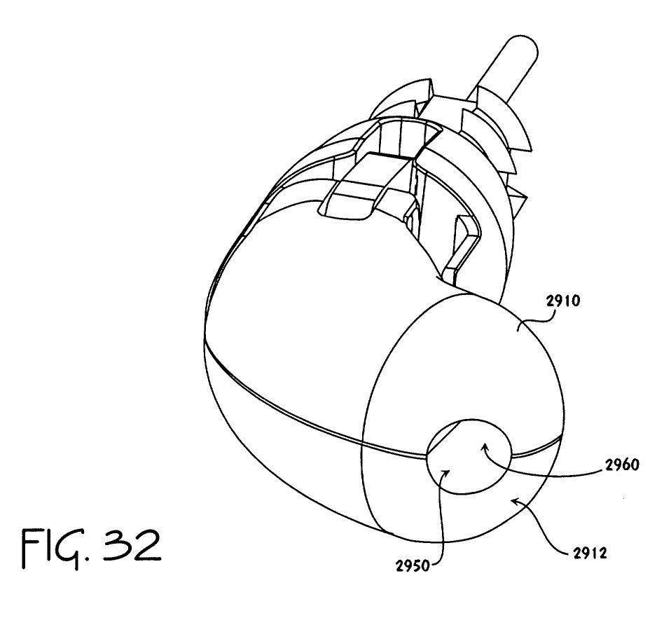

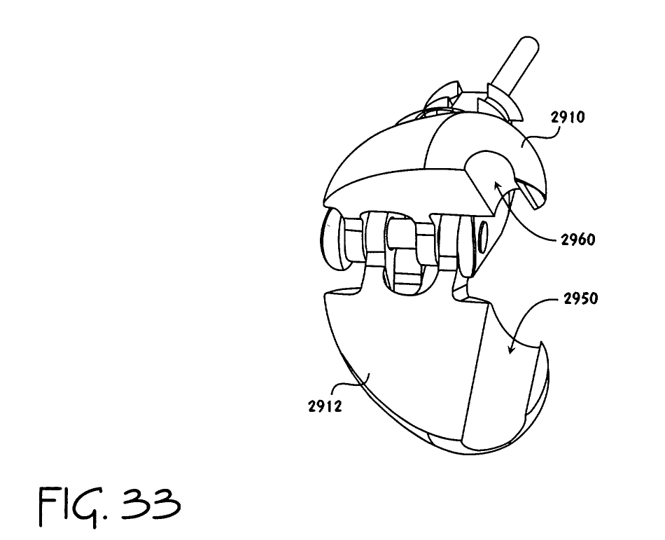

An alternative embodiment, shown in FIGS. 32 and 33, includes tissue expansion members possessing a mutual recessed area forming a lumen or guide wire lumen that is offset from the central axis. Each tissue expansion member (2910, 2912) includes a recessed area on the tissue expansion member's opposing face. The recessed areas (2950, 2960) on the opposing faces of the tissue expansion members (2910, 2912) can be positioned such that when the tissue expansion members (2910, 2912) are in the closed, un-engaged position the recessed areas form a lumen. With the tissue expansion members closed or in the un-engaged position, the apparatus can be guided to the occlusion via a guide wire traversing through the lumen formed by the two recessed areas (2950, 2960). Upon reaching the occlusion and opening the tissue expansion members, the apparatus can be disengaged from the guide wire and utilized to dissect a vascular occlusion without being impeded by a guide wire.

Finally, several features can be common to all embodiments. Referring back to FIG. 1 of the first embodiment but understanding that such features could be included in any embodiment, the retention fins 1128 can be configured to engage the interior distal portion of a catheter tube body. Accordingly, a lumen defined by a standard catheter tube body is placed over the retention fins 1128 coupling the catheter body to the base section 1102. This coupling establishes a linear tensile strength between the jaw assembly and the shaft. One skilled in the relevant art will recognize various methods of achieving such a coupling including chemical and mechanical bonding, friction fitting, spot welding, thermal bonding and the like. The base section 1102 can also include a mounting set channel 1126 used to fuse or bond the interior of the catheter tube body to the base section 1102. Accordingly, the catheter tube body can be heat fused or bonded (e.g., melted) into the space defined by the mounting set channel 1126, providing a secure coupling of the catheter body to the base section 1102. This mode of coupling enhances the rotational torque strength between the jaw assembly and the catheter shaft. Yet, in another aspect of coupling the base section to catheter tube, the base section 1102 includes a smooth coupling surface and does not incorporate a mounting set channel 1126 or retention fins 1128 allowing a catheter tube body to be joined by conventional techniques.

The individual tissue expansion members may have a variety of different configurations, shapes, and sizes including but not limited to spade shaped, straight with a concave curve at the end, straight with convex curve at the end, triangular, rectangular and various combinations thereof. Furthermore, the tissue expansion members can be of different lengths allowing a user of the device to affect the direction of the dissection by rotating the apparatus until the longer jaw is positioned towards the direction which is desired of the dissection plane. These configurations or shapes may be used in combination with each other or separately. Moreover, the individual tissue expansion members may be integrally formed from a single piece of suitable material or may include a combination of different components.

From the above description and drawings, it will be understood by those of ordinary skill in the art that the particular embodiments shown and described are for purposes of illustration only and are not intended to limit the scope of the invention. Those of ordinary skill in the art will recognize that the invention may be embodied in other specific forms without departing from its spirit or essential characteristics. References to details of particular embodiments are not intended to limit the scope of the claims. All of the above references and U.S. patents and applications are incorporated herein by reference.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” and words of similar import, when used in this application, shall refer to this application as a whole and not to any particular portions of this application.

The above detailed descriptions of embodiments of the invention are not intended to be exhaustive or to limit the invention to the precise form disclosed above. While specific embodiments of, and examples for, the invention are described above for illustrative purposes, various equivalent modifications are possible within the scope of the invention, as those skilled in the relevant art will recognize. Furthermore, the elements and acts of the various embodiments described above can be combined to provide further embodiments.

These and other changes can be made to the invention in light of the above detailed description. In general, the terms used in the following claims, should not be construed to limit the invention to the specific embodiments disclosed in the specification, unless the above detailed description explicitly defines such terms. Accordingly, the actual scope of the invention encompasses the disclosed embodiments and all equivalent ways of practicing or implementing the invention under the claims.

While certain aspects of the invention are presented below in certain claim forms, the inventors contemplate the various aspects of the invention in any number of claim forms. Accordingly, the inventors reserve the right to add additional claims after filing the application to pursue such additional claim forms for other aspects of the invention.

We claim:

1. A tissue expansion apparatus comprising:
a base section;
a hinge assembly coupled with the distal region of the base section having two hinge assembly arms extending in the distal direction;
a plurality of tissue expansion members;
an actuating assembly (comprising an actuating member and an actuation plate) for the application of a force perpendicular to the central axis of the base section on a tissue expansion member; and
a coupling system for engaging the tissue expansion members to the actuation plate; and
a coupling system for engaging the tissue expansion members to the base section for simultaneous outward movement of the tissue expansion members relative to the central axis of the base section.

2. The apparatus of claim 1, wherein the coupling system is offset from the central longitudinal axis.

3. The apparatus of claim 1, wherein the base section includes an actuation channel along a longitudinal axis passing completely through the base section on the distal face for accommodating the actuation plate.

4. The apparatus of claim 3, wherein the base section includes a base aperture along the longitudinal axis beginning at the proximal end of the base section and terminating in the actuation channel.

5. The apparatus of claim 4, wherein the base section includes a hinge assembly having a hinge assembly aperture in each hinge assembly arm along the transverse axis with respect to the actuation channel.

6. The apparatus of claim 5, wherein each tissue expansion member includes a plurality of apertures for coupling each tissue expansion member to the hinge assembly and actuation plate.

7. The apparatus of claim 1, wherein the coupling system further comprises a hinge pin enabling the coupling of each tissue expansion member to the hinge assembly and a plurality of coupling pins enabling the coupling of each tissue expansion member to the actuation plate.

8. The apparatus of claim 7, wherein each tissue expansion member is coupled to the hinge assembly arms at the hinge assembly aperture by the hinge pin passing transverse to the central axis of the apparatus.

9. The apparatus of claim 8, wherein the actuating member is coupled to the actuation plate.

10. The apparatus of claim 9, wherein the actuation plate includes a plurality of actuation plate apertures.

11. The apparatus of claim 10, wherein the actuation plate apertures are oval.

12. The apparatus of claim 11, wherein each tissue expansion member is coupled to the actuating plate at said actuation plate aperture by a coupling pin.

13. The apparatus of claim 9, wherein the actuating member is fixed to the actuation plate.

14. The apparatus of claim 1, wherein the base section includes retention fins enabling a coupling of a catheter tube to the base section.

15. The apparatus of claim 1, wherein the base section includes a mounting set channel enabling a coupling of a catheter tube to the base section.

16. The apparatus of claim 1, wherein the actuating member and actuation plate are placed within the base aperture and actuation channel respectively allowing movement of the actuation assembly within said base section.

17. The apparatus of claim 1, wherein the shape of the tissue expansion members is modifiable.

18. A method of vascular micro-dissection comprising;
placing a tissue expansion apparatus in the proximity of a vascular occlusion wherein the tissue expansion apparatus comprises a base section, a hinge assembly coupled with distal region of the base section having two hinge assembly arms extending in the distal direction, an actuating assembly, and a plurality of tissue expansion members, wherein each actuation assembly includes an actuating member and an actuation plate for the application of a force perpendicular to the central axis of the base section on each tissue expansion member;
coupling the tissue expansion members to the base section;
coupling the tissue expansion members to the actuating assembly;
applying a force along the longitudinal axis of the tissue expansion apparatus to the actuating assembly, wherein the force simultaneously rotates the tissue expansion members outward relative to the longitudinal axis of the tissue expansion apparatus; and
disrupting the vascular occlusion in response to the force.

19. The method of claim 18, further comprising enabling the passage of a guide-wire or other intervention device through a dissection tract produced by the tissue expansion apparatus.

20. The method of claim 18, wherein disrupting includes tearing the vascular occlusion.

21. The method of claim 18, wherein disrupting includes fracturing the vascular occlusion.

22. The method of claim 18, wherein disrupting includes separating the vascular occlusion from the vasculature wall.

23 The method of claim 18, wherein disrupting includes separating vessel wall tissue and creating a dissection tract within the vessel wall.

24. The method of claim 18, further comprising placing the tissue expansion apparatus near the occlusion by guiding the tissue expansion apparatus via a guide-wire.

25. The method of claim 18, wherein the coupling further comprises placing the actuation assembly within the base section allowing longitudinal movement of the actuating assembly relative to the base section.

26. The method of claim 18, wherein placing the tissue expansion apparatus further comprises the coupling of the tissue expansion apparatus to a catheter tube.
